(19) United States
(12) Patent Application Publication
(10) Pub. No.: US 2022/0306986 A1
(43) Pub. Date: Sep. 29, 2022

(54) NON-INVASIVE METHODS FOR SELECTIVELY ENRICHING PLURIPOTENT CELLS

(71) Applicant: MEMORIAL SLOAN-KETTERING CANCER CENTER, New York, NY (US)

(72) Inventors: Lydia W.S. Finley, New York, NY (US); Craig B. Thompson, New York, NY (US); Santosha A. Vardhana, Brooklyn, NY (US)

(73) Assignee: MEMORIAL SLOAN-KETTERING CANCER CENTER, New York, NY (US)

(21) Appl. No.: 17/514,742

(22) Filed: Oct. 29, 2021

Related U.S. Application Data
(63) Continuation of application No. PCT/US2020/030703, filed on Apr. 30, 2020.
(60) Provisional application No. 62/840,956, filed on Apr. 30, 2019.

(51) Int. Cl. C12N 5/0735

(52) U.S. Cl. CPC C12N 5/0606; C12N 2506/03; C12N 2500/44; C12N 2501/235; C12N 2500/24; C12N 2501/115; C12N 2500/32

(57) ABSTRACT

The present disclosure provides highly efficient, non-invasive, and reversible methods for selectively enriching pluripotent cells (e.g., human pluripotent cells and mouse pluripotent cells) in a cell population using a glutamine-deficient medium. The presently disclosed methods have the advantageous of efficiently enriching pluripotent cells in a heterogenous cell population without altering the biological properties of any individual cells.

### CROSS-REFERENCE TO RELATED APPLICATIONS

This application is a Continuation of International Patent Application No. PCT/US2020/030703, filed Apr. 30, 2020, which claims priority to U.S. Provisional Patent Application Ser. No. 62/840,956, filed Apr. 30, 2019, the contents of which are incorporated by reference in their entireties, and to each of which priority is claimed.

### GRANT INFORMATION

This invention was made with government support under CA191021 and CA008748 and awarded by National Institutes of Health. The government has certain rights in the invention.

### SEQUENCE LISTINGS

The instant application contains a Sequence Listing which has been submitted electronically in ASCII format and is hereby incorporated by reference in its entirety. Said ASCII copy, created on Oct. 28, 2021, is named 072734_1284_ST25.txt and is 3,066 bytes in size.

### 1. INTRODUCTION

The present disclosure relates to highly efficient, non-invasive, and reversible methods for selectively enriching pluripotent cells (e.g., human pluripotent cells and mouse pluripotent cells) in a heterogenous cell population using a glutamine-deficient medium, and kits and compositions relating thereto.

### 2. BACKGROUND

When induced to proliferate in culture, mammalian cells rewire metabolic pathways to support the anabolic demands of cell growth. Cells take up high levels of glucose and glutamine, which are used to generate the building blocks, reducing equivalents and energy required to duplicate biomass prior to cell division (DeBerardinis et al., Cell metabolism 7, 11-20 (2008)). Consequently, exogenous supplies of both glucose and glutamine are essential to sustain rapid proliferation of most cultured cell lines (DeBerardinis et al., Cell metabolism 7, 11-20 (2008)). While proliferating cells of all lineages share many common metabolic features, most notably elevated glycolysis and glutaminolysis, recent evidence demonstrates that there is not one single mode of proliferative metabolism. Rather, cells can engage multiple routes of nutrient acquisition and catabolism to support survival and proliferation (Palm et al., Nature 546, 234-242 (2017)). Several factors contribute to this metabolic diversity, including cell lineage, genetic makeup and environmental conditions (Vander Heiden et al., Cell 168, 657-669 (2017)). This raises the intriguing possibility that metabolic manipulation can provide selective pressures that promote or antagonize the proliferation of distinct cell types in a predictable manner.

Metabolites serve many roles beyond anabolic building blocks. Metabolites also serve as signals or effectors that affect myriad cellular processes, including signal transduction, stress response pathways and chemical modification of proteins and nucleic acids (Schvartzman et al., The Journal of cell biology 217, 2247-2259 (2018); Saxton et al., Cell 168, 960-976, (2017)). Consequently, regulation of cellular metabolism has emerged as a mechanism to influence cell fate decisions beyond proliferation. In particular, many of the enzymes that modify DNA and histones require metabolites as necessary co-substrates, raising the possibility that metabolic fluctuations shape the chromatin landscape and, in turn, affect gene expression programs (Schvartzman et al., The Journal of cell biology 217, 2247-2259 (2018); Su et al., Curr Opin Chem Biol 30, 52-60, (2016)). Indeed, pathological accumulation of certain metabolites in many malignancies is sufficient to block differentiation and promote transformation by disrupting the normal dynamic chromatin regulation of progenitor cells (Lu et al., Cell metabolism 16, 9-17 (2012)).

Collectively, these findings suggest that how a cell solves the problem of proliferative metabolism may have consequences for the regulation of cell identity. The link between proliferation and cell identity is especially critical in pluripotent stem cells, which proliferate rapidly in culture while retaining the capacity to differentiate into all three lineages of the developing embryo. Pluripotent stem cells utilize glucose and glutamine to fuel proliferation, and perturbations in the metabolism of these nutrients can alter both survival and differentiation. Notably, glucose-derived acetyl-CoA, the substrate for histone acetyltransferases, and glutamine derived α-ketoglutarate (αKG), a co-substrate of αKG-dependent dioxygenases including the Tet family of methylcytosine oxidases and the Jumonji-domain containing family of histone demethylases, contribute to the regulation of the chromatin landscape, thereby influencing the balance of self-renewal vs differentiation (Carey et al., Nature 518, 413-416 (2015); Hwang et al., Cell metabolism 24, 494-501 (2016); Moussaieff et al., Cell metabolism 21, 392-402, (2015); TeSlaa et al., Cell metabolism 24, 485-493 (2016)).

### 3. SUMMARY OF THE INVENTION

The present disclosure provides highly efficient, non-invasive and reversible methods for selectively enriching pluripotent cells (e.g., human pluripotent cells and mouse pluripotent cells) in a cell population using a glutamine-deficient medium. It is based on the discovery that cells with weak pluripotency-associated transcription networks are highly glutamine dependent and rapidly die in the absence of exogenous glutamine supplementation.

In one aspect, the present disclosure provides a method for selectively enriching pluripotent cells in a cell population comprising non-pluripotent cells and the pluripotent cells, wherein the method comprises culturing the cell population in a glutamine-deficient medium. In certain embodiments, the pluripotent cells are self-renewing pluripotent cells.

In one aspect, the present disclosure provides a method for selectively enriching fully reprogrammed pluripotent cells in a cell population comprising not fully reprogrammed cells and the fully reprogrammed pluripotent cells, wherein the method comprises culturing the cell population in a glutamine-deficient medium.

In certain embodiments, the cell population are derived from somatic cells, wherein the somatic cells have been subject to reprogramming to induce acquired pluripotency. In certain embodiments, the cell population is cultured in the glutamine-deficient medium transiently.

In certain embodiments, the cell population is cultured in the glutamine-deficient medium for between about 4 hours and about 48 hours. In certain embodiments, the cell population is cultured in the glutamine-deficient medium for about 24 hours. In certain embodiments, the method further comprises culturing the cell population in a complete medium comprising glutamine. In certain embodiments, the method further comprises culturing the cell population in the complete medium after culturing the cell population in the glutamine-deficient medium. In certain embodiments, the cell population is cultured in the complete medium for at least about 24 hours. In certain embodiments, the cell population is cultured in the complete medium for about 48 hours.

In certain embodiments, the level of the pluripotent cells or the fully reprogrammed pluripotent cells is increased between about 10% to about 500% as compared to the level of pluripotent cells or fully reprogrammed pluripotent cells in a cell population that has not been cultured in the glutamine-deficient medium. In certain embodiments, the pluripotent cells or the fully reprogrammed pluripotent cells are selectively enriched in the cell population to a level of about 98%, 99%, or 100% of the cell population.

In certain embodiments, the pluripotent cells has an elevated cellular αKG/succinate ratio as compared to the non-pluripotent cells. In certain embodiments, the pluripotent cells express a high level of Nanog, Oct4, Sox2, Esrrb, Zfp42, Klf4, Tfcp2l1, Stat3, or combinations thereof as compared to the non-pluripotent cells. In certain embodiments, the fully reprogrammed pluripotent cells express a high level of Nanog, Oct4, Sox2, Esrrb, Zfp42, Klf4, Tfcp2l1, Stat3, or combinations thereof as compared to the not fully reprogrammed cells.

In one aspect, the present disclosure provides a plurality of pluripotent cells, wherein the pluripotent cells are selectively enriched in a cell population comprising non-pluripotent cells and the pluripotent cells, after culturing the cell population in a glutamine-deficient medium.

In certain embodiments, the pluripotent cells are self-renewing pluripotent cells. In certain embodiments, the cell population is cultured in the glutamine-deficient medium transiently. In certain embodiments, the cell population is cultured in the glutamine-deficient medium for between about 4 hours and about 48 hours. In certain embodiments, the cell population is cultured in the glutamine-deficient medium for about 24 hours.

In certain embodiments, the pluripotent cells further comprise the cell population is cultured in a complete medium comprising glutamine. In certain embodiments, the pluripotent cells further comprise the cell population is cultured in the complete medium after culturing the cell population in the glutamine-deficient medium. In certain embodiments, the cell population is cultured in the complete medium for at least about 24 hours. In certain embodiments, the cell population is cultured in the complete medium for about 48 hours.

In certain embodiments, the level of the pluripotent cells in the cell population is increased between about 10% to about 500% as compared to the level of pluripotent cells in a cell population that has not been cultured in the glutamine-deficient medium. In certain embodiments, the pluripotent cells are selectively enriched in the cell population to a level of about 98%, 99%, or 100% of the cell population.

In certain embodiments, the pluripotent cells has an elevated cellular αKG/succinate ratio as compared to the non-pluripotent cells. In certain embodiments, the pluripotent cells express a high level of Nanog, Oct4, Sox2, Esrrb, Zfp42, Klf4, Tfcp2l1, Stat3, or combinations thereof, as compared to the non-pluripotent cells.

In one aspect, the present disclosure provides a plurality of fully reprogrammed pluripotent cells, wherein the fully reprogrammed pluripotent cells are selectively enriched in a cell population comprising not fully reprogrammed cells and the fully reprogrammed pluripotent cells, after culturing the cell population in a glutamine-deficient medium. In certain embodiments, the cell population are derived from somatic cells, wherein the somatic cells have been subject to reprogramming to induce acquired pluripotency.

In certain embodiments, the cell population is cultured in the glutamine-deficient medium transiently. In certain embodiments, the cell population is cultured in the glutamine-deficient medium for between about 4 hours and about 48 hours. In certain embodiments, the cell population is cultured in the glutamine-deficient medium for about 24 hours.

In certain embodiments, the plurality of fully reprogrammed pluripotent cells further comprises the cell population is cultured in a complete medium comprising glutamine. In certain embodiments, the plurality of fully reprogrammed pluripotent cells further comprises the cell population is cultured in the glutamine-deficient medium. In certain embodiments, the cell population is cultured in the complete medium for at least about 24 hours. In certain embodiments, the cell population is cultured in the complete medium for about 48 hours.

In certain embodiments, the level of the fully reprogrammed pluripotent cells in the cell population is increased between about 10% to about 500% as compared to the level of fully reprogrammed pluripotent cells in a cell population that has not been cultured in the glutamine-deficient medium. In certain embodiments, the fully reprogrammed pluripotent cells are selectively enriched in the cell population to a level of about 98%, 99%, or 100% of the cell population.

In certain embodiments, the fully reprogrammed pluripotent cells express a high level of Nanog, Oct4, Sox2, Esrrb, Zfp42, Klf4, Tfcp2l1, Stat3, or combinations as compared to the not fully reprogrammed cells.

In one aspect, the present disclosure provides a composition comprising the pluripotent cells disclosed herein.

In one aspect, the present disclosure provides a composition comprising the full programmed pluripotent cells disclosed herein.

In one aspect, the present disclosure provides a kit for selectively enriching pluripotent cells, comprising: a glutamine-deficient medium, and a cell population comprising non-pluripotent cells and the pluripotent cells. In certain embodiments, the pluripotent cells are self-renewing pluripotent cells. In one aspect, the present disclosure provides a kit for selectively enriching fully reprogrammed pluripotent cells, comprising a glutamine-deficient medium, and a cell population comprising not fully reprogrammed cells and the fully reprogrammed pluripotent cells.

In certain embodiments, the cell population is derived from somatic cells, where the somatic cells have been subject to reprogramming to induce acquired pluripotency.

In certain embodiments, the kit further comprises instructions for selectively enriching the pluripotent cells or the fully reprogrammed pluripotent cells, wherein the instructions comprises culturing the cell population in the glutamine-deficient medium.

In certain embodiments, the instructions comprises culturing the cell population in the glutamine-deficient medium transiently. In certain embodiments, the instructions comprises culturing the cell population in the glutamine-deficient medium for between about 4 hours and about 48 hours. In certain embodiments, the instructions comprises culturing the cell population in the glutamine-deficient medium for about 24 hours.

In certain embodiments, the kit further comprises a complete medium comprising glutamine. In certain embodiments, the instructions comprises culturing the cell population in the complete medium. In certain embodiments, the instructions comprises culturing the cell population in the complete medium after culturing the cell population in the glutamine-deficient medium. In certain embodiments, the instructions comprises culturing the cell population in the complete medium for at least about 24 hours. In certain embodiments, the instructions comprises culturing the cell population in the complete medium for about 48 hours.

### 4. BRIEF DESCRIPTION OF THE DRAWINGS

FIGS. 1A-1J show glutamine anaplerosis was reduced in eSCs with enhanced self-renewal. FIGS. 1A and 1C are schematics depicting how oxidative metabolism of uniformly labelled glucose ([U-¹³C]glucose) (FIG. 1A) and glutamine ([U-¹³C] glutamine) (FIG. 1C) generates metabolites associated with the TCA cycle. The circles represent ¹³C-labelled carbons. FIGS. 1B and 1E show fractional m+2 labelling of citrate, αKG, glutamate, fumarate, malate and aspartate in ESCs expressing the G-CSF-activated LIF receptor transgene cultured with or without G-CSF (FIG. 1B) or ESCs expressing empty vector, Klf4 or Nanog (FIG. 1E) cultured in medium containing [U-¹³C] glucose. FIGS. 1D and 1F show fractional m+5 labelling of glutamate and αKG and m+4 labelling of fumarate, malate, aspartate and citrate in ESCs expressing the G-CSF-activated LIF receptor transgene cultured with or without G-CSF (FIG. 1D) or ESCs expressing empty vector, Klf4 or Nanog (FIG. 1F) cultured in medium containing [U-¹³C] glutamine. FIGS. 1G and 1H depict quantification of the αKG/succinate ratio in ESCs expressing the G-CSF-activated LIF receptor transgene cultured with or without G-CSF (FIG. 1G) or ESCs expressing empty vector, Klf4 or Nanog (FIG. 1H). FIG. 1I shows separation of Nanog^{low} and Nanog^{high} populations by FACS. Left, shaded grey represents the parental Nanog-GFP population cultured in serum/LIF medium; the top 10% and bottom 10% populations were sorted and plated for subsequent experiments. Right, flow cytometry analysis of Nanog^{low} and Nanog^{high} populations 72 h after initial sorting. FIG. 1J shows quantification of the αKG/succinate ratio in the Nanog^{low} and Nanog^{high} populations shown in panel FIG. 1I (right). The experiment was repeated independently twice with similar results. P values were calculated using an unpaired, two-sided Student's t-test (FIGS. 1B, 1D, 1G, and 1I) or a one-way analysis of variance (ANOVA) with Sidak's multiple comparisons post-hoc test (FIGS. 1E, 1F, and 1H) relative to control cells (FIGS. 1B, 1D, and 1E-1H) or Nanog^{low} cells (FIG. 1J). Data are presented as the mean±s.d. of n=3 biologically independent samples from a representative experiment (FIGS. 1B, 1D, 1E-1H, and 1J).

FIGS. 2A-2L show enhanced self-renewal improves glutamine-independent survival. FIG. 2A shows alkaline phosphatase staining of colony formation assays of ESCs subjected to glutamine withdrawal for the indicated times in the presence of DMSO (Vehicle) or dimethyl-αKG (DM-αKG). One representative well is shown. The experiment was performed independently twice with similar results. FIG. 2B shows quantification of apoptosis in Nanog^{high} and Nanog^{low} ESCs 72 h after sorting based on Nanog-GFP expression. Cells were deprived of glutamine for the final 24 h. FIGS. 2C and 2D depict viability (measured by DAPI exclusion) (FIG. 2C) or population doublings (FIG. 2D) of ESCs expressing G-CSF-activated LIF receptor transgene cultured with or without G-CSF and deprived of glutamine for 48 h. FIG. 2E shows population doublings of ESCs expressing empty vector, Klf4 or Nanog during 48 h of culture in glutamine-free medium. FIGS. 2F-2H show population doublings of ESCs cultured for 48 h in glutamine-free medium unless otherwise noted. Where noted, the following compounds were included: JAKi, (ruxolitinib, 500 nM); DM-αKG (4 mM); dimethyl-succinate (DM-succinate, 4 mM); methyl-pyruvate (2 mM); or glutamine synthetase inhibitor methionine sulfoximine (MSO; 1 mM). FIGS. 2I and 2J depict quantification of the αKG/succinate ratio in ESCs expressing the G-CSF-activated LIF receptor transgene cultured with or without G-CSF following 8 h of glutamine deprivation (FIG. 2I) or ESCs expressing empty vector, Klf4 or Nanog following 4 h of glutamine deprivation (FIG. 2J). FIG. 2K shows fraction of Nanog-GFP ESCs expressing the G-CSF-activated LIF receptor transgene cultured with or without G-CSF exhibiting high Nanog-GFP expression after culture in the presence or absence of glutamine for 48 h. FIG. 2L shows Relative accumulation of GFP⁺ ESCs expressing empty vector, Klf4 or Nanog compared to parental controls following culture in the presence or absence of 2 mM glutamine for 48 h. The ratio represents the fraction of GFP⁺ cells after culture minus glutamine relative to the fraction of GFP⁺ cells after culture plus glutamine. P values were calculated using an unpaired, two-sided Student's t-test (FIGS. 2C, 2D, 2F-2I, and 2K) or a one-way ANOVA with Sidak's multiple comparisons post-hoc test (FIGS. 2B, 2E, 2J, and 2L), relative to Nanog^{low} cells (FIG. 2B), control ESCs (FIGS. 2C-2E, and 2I-2L) or cells cultured in glutamine-deficient medium alone (FIGS. 2F-2H). Data are presented as the mean±s.d. of n=3 biologically independent samples from a representative experiment (FIGS. 2B-2L).

FIGS. 3A-3I show transient glutamine withdrawal enhances eSC self-renewal. FIG. 3A shows quantification of Oct4 immunofluorescence in ESCs cultured in the absence of glutamine for the indicated times. Dashed line denotes threshold for Oct4^{low} cells, defined as one standard deviation below the mean values of the control population. FIG. 3B shows experimental design for transient glutamine withdrawal (Pulse—glutamine). Final analyses, including fixation of colony formation assays (CFA), were performed at the indicated times after D3. FIG. 3C shows quantification of Oct4 and Nanog immunofluorescence in control (Ctrl) or serum/LIF+2i (2i)-cultured ESCs or ESCs previously subjected to 24 h of glutamine deprivation (Pulse—glutamine). “a.u.” represents “arbitrary unit”. FIG. 3D shows expression of Nanog-GFP in ESC subjected to glutamine withdrawal for 24 h and then recovered with glutamine-replete medium for 24 h (Pulse—glutamine) or maintained in glutaminereplete medium (Ctrl). FIG. 3E shows alkaline phosphatase staining of colony formation assays where two different ESC lines were maintained in glutamine-replete medium (Ctrl) or subjected to transient glutamine withdrawal for 24 h and then recovered in glutamine-replete medium for 24 h prior to plating (Pulse—glutamine). One representative well is shown. FIG. 3F shows quantification of colonies formed in (FIG. 3E). Colonies were scored manually as undifferentiated, mixed or differentiated based on alkaline phosphatase staining. FIGS. 3G-3I show quantification of colony formation assays where ESCs were maintained continuously in serum/LIF medium containing glutamine (Ctrl) or subjected to 24 h of glutamine withdrawal followed by recovery in control medium for 24 h before plating (Pulse—glutamine). Additional manipulations included exposing cells to 2i continuously (2i continuous) or for 24 h (Pulse 2i) (FIG. 3G), the addition of 4 mM DM-αKG or 1 mM MSO during the ‘pulse’ (FIG. 3H) and transient withdrawal of glutamine and/or glucose for 24 h followed by recovery in complete medium for 24 h before plating (i). P values were calculated using unpaired, two-sided Student's t-tests (FIG. 3F) or one-way ANOVA with Sidak's multiple comparisons post-hoc test (FIGS. 3G-3I), relative to control ESCs. Data are presented as the mean±s.e.m. (FIG. 3F) or s.d. (FIGS. 3G-3I) of n=6 biologically independent samples from a representative experiment, or as >10,000 cells pooled from n=3 biologically independent samples from a representative experiment (FIGS. 3A and 3C). The experiment was repeated independently twice (FIGS. 3A, 3C, and 3E) or three times (FIG. 3D) with similar results.

FIGS. 4A-4G show transient glutamine withdrawal improves mouse somatic cell reprogramming to pluripotency and enhances human eSC self-renewal. FIG. 4A shows experimental design for the reprogramming of MEFs expressing DOX-inducible Oct4, Sox2, Klf4 and c-Myc (OKSM). Cells were subjected to DOX for 8 d. On day 10, cells were exposed to 2i for the duration of the experiment (+2i), 24 h of glutamine deprivation (Pulse—glutamine), 24 h of 2i (Pulse 2i) or maintained in glutamine-replete medium (Ctrl). FIG. 4B shows alkaline phosphatase staining of a representative well of cells reprogrammed as described in (FIG. 4A). FIG. 4C shows quantification of the number of round, highly-alkaline phosphatase-stained colonies representing successfully reprogrammed colonies formed from OKSM-MEFs 14 d after initial DOX addition. FIG. 4D shows experimental design for reprogramming of Oct4-GFP MEFs. Cells were infected with the OKSM virus the day after seeding. The following day, cells began 12 d of DOX exposure. On day 14, cells were exposed to 2i for the duration of the experiment (+2i), 24 h of glutamine deprivation (Pulse—glutamine), 24 h of 2i (Pulse 2i) or maintained in glutamine-replete medium (Ctrl). FIG. 4E shows percentage of Oct4-GFP-expressing cells as an indicator of successful reprogramming at day 21 following initial DOX induction. FIG. 4F shows expression of OCT4 and SOX2 in human ESCs subjected to glutamine withdrawal for 24 h and then recovered with glutamine-replete medium for 24 h (Pulse—glutamine) or maintained in glutamine-replete medium (Ctrl). FIG. 4G shows quantification of OCT4 and SOX2 MFI as well as percentage of OCT4/SOX2^{high} cells as depicted in FIG. 4F. P values were calculated using an unpaired, two-sided Student's t-test (FIG. 4G) or a one-way ANOVA with Sidak's multiple comparisons post-hoc test (FIGS. 4C and 4E) relative to control ESCs maintained in glutamine-replete medium. Data are presented as the mean±s.d. of n=6 (FIGS. 4C and 4E) or n=3 (FIG. 4G) biologically independent samples from a representative experiment.

FIGS. 5A-5G show enhancing ESC self-renewal leads to decreased glutamine anaplerosis. FIGS. 5A and 5B show immunoblot of phospho-STAT3 and total STAT3 (FIG. 5A) or qRT-PCR of STAT3-target genes and other key pluripotency genes (FIG. 5B) in ESCs expressing GCSF-activated LIF receptor transgene cultured with or without GCSF. FIG. 5C shows quantification of glutamate pools in ESCs expressing GCSF-activated LIF receptor transgene cultured with or without GCSF in medium containing [U-¹³C]glucose and following 4 h of glutamine withdrawal. Quantification of pools either labeled or not labeled by glucose-derived [U-¹³C] are indicated. FIGS. 5D and 5E show immunoblot of Nanog and Klf4 (FIG. 5D) or qRT-PCR of key pluripotency genes (FIG. 5E) in ESCs expressing empty vector, Klf4, or Nanog. FIG. 5F shows schematic depicting how metabolism of uniformly-labeled glucose ([U-¹³C] glucose) via pyruvate carboxylase generates metabolites associated with the TCA cycle. Colored circles represent ¹³C-labeled carbons. FIGS. 5G and 5H show fractional m+3 labeling of aspartate (Asp), malate (Mal) and fumarate (Fum) in ESCs expressing GCSF-activated LIF receptor transgene cultured with or without GCSF (FIG. 5G) or ESCs expressing empty vector, Klf4 or Nanog (FIG. 5H) cultured in medium containing [U-¹³C]glucose. FIGS. 5I and 5J show population doublings of ESCs expressing GCSF-activated LIF receptor transgene cultured with or without GCSF (i) or ESCs expressing empty vector, Klf4 or Nanog (FIG. 5J) during 48 h of culture in glutamine-replete medium. FIG. 5K shows quantification of the αKG/succinate ratio in Nanog Low, Nanog Medium, and Nanog High populations. Nanog-GFP ESCs cultured in S/L medium were sorted based on GFP expression; the top 10%, bottom 10%, and median 10% populations were sorted and cultured for 48 h prior to harvesting for metabolite extraction and analysis. P values were calculated by unpaired, two-sided Student's t-test (FIGS. 5B, 5C, 5G, and 5I) or one-way ANOVA with Sidak's multiple comparisons post-test (FIGS. 5E, 5H, 5J, and 5K), relative to control ESCs (FIGS. 5B, 5C, 5E, 5G, 5H, 5I, and 5J) or Nanog Low ESCs (FIG. 5K). Data are presented as the mean±s.d. of n=3 biologically independent samples from a representative experiment (FIGS. 5B, 5C, 5G, 5H, 5I, 5J, and 5K). Experiments were repeated 2 times with similar results (FIGS. 5A and 5D). Molecular weight marker (in kDa) is shown (FIGS. 5A and 5D).

FIGS. 6A-6K show glutamine is a major source of TCA cycle anaplerosis in ESCs. FIG. 6A depicts population doublings of ESCs during 72 h of culture in medium containing or lacking glutamine as indicated. FIG. 6B depicts viability of ESCs after 24 hours of culture in medium containing glutamine, with or without the addition of 4 mM cell-permeable dimethyl-αketoglutarate (DM-αKG) as measured by DAPI exclusion. FIG. 6C depicts quantification of viability in Nanog High, Nanog medium, and Nanog Low ESCs sorted based on Nanog-GFP expression and deprived of glutamine for 24 h. All cells were recovered in medium 2 mM glutamine for 24 h prior to culturing in media lacking glutamine. Viability measured as percentage of cells excluding DAPI. FIG. 6D depicts median Nanog-GFP expression measured by flow cytometry in Nanog High and Nanog Low ESCs (described in FIG. 2A) 72 h after sorting based on Nanog-GFP expression and deprived of glutamine for the final 24 h. Unsorted cells cultured in S/L+2i are included as a control. FIG. 6E depicts population doublings of ESCs during 48 h of culture in medium containing glutamine, with or without the addition of 4 mM cell-permeable dimethyl-a ketoglutarate (DM-αKG), 4 mM dimethyl-succinate (DM-succ), or 2 mM methyl-pyruvate (me-pyruvate) as indicated. FIG. 6F depicts population doublings of ESCs during 48 h of culture in medium containing glutamine, with or without the addition of 4 mM cell-permeable dimethyl-a ketoglutarate (DM-αKG) and with or without the addition of 200 nM methionine sulfoximine (MSO) as indicated. FIG. 6G depicts relative abundance of intracellular TCA cycle metabolites following 8 h of glutamine withdrawal in ESCs expressing GCSF-activated LIF receptor transgene cultured with or without GCSF relative to control ESCs cultured in glutamine-replete medium. FIG. 6H depicts relative abundance of intracellular TCA cycle metabolites following 8 h of glutamine withdrawal in ESCs expressing empty vector, Klf4 or Nanog relative to empty vector ESCs cultured in glutamine-replete medium. FIG. 6I depicts quantification of intracellular α-KG pools, in ESCs expressing GCSF-activated LIF receptor transgene cultured with or without GCSF in medium lacking glutamine for 4 hours. FIG. 6J depicts quantification of intracellular α-KG pools, in ESCs expressing empty vector, Klf4 or Nanog cultured in medium lacking glutamine for 4 hours. FIG. 6K depicts median Nanog-GFP expression in ESCs expressing GCSF-activated LIF receptor transgene cultured with or without GCSF and grown in the presence or absence of glutamine for 48 h. P values were calculated by unpaired, two-tailed Student's t-test (FIGS. 6A, 6G, 6I, and 6K) or one-way ANOVA with Sidak's multiple comparisons post-test (FIGS. 6C, 6D, 6H, and 6J). Data are presented as the mean±s.d. of triplicate wells from a representative experiment.

FIGS. 7A-7J show quantification of immunofluorescence staining of Oct4 and Nanog. FIG. 7A depicts quantification of nuclear Nanog (left) and Oct4 (right) immunofluorescence in ESCs cultured in either S/L (grey) or S/L+2i (blue) medium. Data are pooled from triplicate wells, n>10,000 cells per condition. Dashed line denotes an estimated threshold for “Nanog-low” and “Oct4 low” cells, respectively, defined as cells as one standard deviation below the mean values of the control population (here, S/L). FIG. 7B depicts quantification of Nanog immunofluorescence in ESCs cultured in the absence of glutamine for the indicated times. Data are pooled from triplicate wells, n>10,000 cells per condition. Dashed lines denotes estimated thresholds for “Nanog low” cells, defined as cells 0.25 standard deviations below the mean values of the control population, and “Nanog high” cells, defined as cells one standard deviation above the mean values of the control population. FIG. 7C depicts expression of Nanog-GFP in ESCs subjected to 24 h of glutamine withdrawal (Withdrawal/−Q), subjected to 24 h of glutamine withdrawal followed by 2 h of culture in glutamine-replete medium (Pulse/−Q), or maintained continuously in standard glutamine-replete medium (+Q). FIG. 7D depicts teratoma formation from ES cells grown either in glutamine-replete S/L medium (Control), subjected to 24 hours of glutamine deprivation followed by recovery with media containing glutamine for 24 hours (Pulse), or adapted to S/L+2i medium for 3 passages (2i). Representative images of haematoxylin and eosin staining revealing differentiation into ectoderm, mesoderm, and endoderm-derived tissue. Scale bar, 50 μM. FIG. 7E depicts expression of Nanog-GFP in ESCs subjected to 24 hours of glutamine withdrawal (Pulse −Q) or 24 hours of S/L media containing 2i (Pulse 2i) and then recovered with glutamine-replete S/L media for 24 hours or maintained continuously in either S/L media containing glutamine (Control) or S/L media containing glutamine and 2i (2i continuous). FIG. 7F depicts expression of Nanog-GFP in ESCs subjected to 24 hours of glutamine withdrawal (Pulse −Q) or 24 hours of S/L media containing 4 mM DM-αKG (Pulse α-KG) and then recovered with glutamine-replete S/L media for 24 hours. FIG. 7G depicts alkaline phosphatase (AP) staining of colony formation assays in which ESCs subjected to 24 hours of glutamine withdrawal (Pulse −Q) or 24 hours of S/L media containing 4 mM DM-αKG (Pulse α-KG) and then recovered with glutamine-replete S/L media for 24 hours or maintained continuously in S/L media containing glutamine (Control), prior to plating at single cell density. One representative well of a six-well plate is shown. FIG. 7H depicts expression of Nanog-GFP in ESCs subjected to 24 h of glutamine withdrawal (Pulse) or maintained in standard glutamine-replete medium (Control) in the presence or absence of methionine sulfoximine (MSO) or the H3K27me3 demethylase inhibitor GSK-J4 and then recovered with glutamine-replete, inhibitor-free medium for 24 hours. FIG. 7I depicts alkaline phosphatase (AP) staining of colony formation assays in which ESCs subjected to 24 h of glutamine withdrawal (Pulse) or maintained in standard glutamine-replete medium (Control) in the presence or absence of GSK-J4 and then recovered with glutamine-replete, inhibitor-free medium for 24 hours, prior to plating at single cell density. One representative well of a six-well plate is shown. FIG. 7J depicts expression of Nanog-GFP in ESCs subjected to withdrawal of either glutamine, glucose, or both glutamine and glucose for 24 hours and then recovered with glutamine and glucose-replete medium for 24 hours or maintained in glutamine and glucose-replete medium as indicated. P values were calculated by unpaired, two-tailed Student's t-test (FIG. 7E) or one-way ANOVA with Sidak's multiple comparisons post-test (FIGS. 7F and 7J). Data are presented as the mean±s.d. of triplicate wells from a representative experiment.

FIGS. 8A-8F show induced pluripotent stem cells (iPSCs) retain both self-renewal and differentiation capacity. FIG. 8A depicts alkaline phosphatase (AP) staining of a representative well of mouse embryonic fibroblasts (MEFs) expressing doxycycline (dox)-inducible Oct4, Sox2, Klf4 and c-Myc (OSKM), either treated with (+dox) or without (−dox) doxycycline for 8 days, followed by culture in S/L medium without dox for 6 days. FIG. 8B depicts percentage of Oct4-GFP-expressing cells as an indicator of successful reprogramming on day 15 (immediately following 24 h of glutamine withdrawal or 24 h of incubation in control media), and day 16 (following 24 h of recovery in glutamine-replete medium). FIG. 8C depicts alkaline phosphatase (AP) staining of a representative well of cells reprogrammed as described in FIG. 4e. FIG. 8D depicts alkaline phosphatase (AP) staining of colony formation assays in which successfully reprogrammed OKSM MEFs were plated at single cell density in the presence or absence of LIF. One representative well of a six-well plate is shown. FIG. 8E depicts quantification of the number of AP stained colonies formed by successfully reprogrammed MEFs in the presence of LIF shown in FIG. 8D. FIG. 8F depicts qRT-PCR of pluripotency associated (Nanog, Esrrb, Rex1) and epiblast-associated (Fgf5) genes in successfully reprogrammed OKSM MEFs cultured in the absence of LIF. P values were calculated by unpaired, two-tailed Student's t-test (FIG. 8B). Data are presented as the mean±s.d. of triplicate wells from a representative experiment.

FIG. 9 depicts gating strategy for fluorescence activated cell sorting analysis. For both Nanog-GFP and Oct4-GFP cell lines, gating was performed from left to right as shown. First, doublet exclusion was performed on cells gated by FSC-H versus FSC-W. Then, doublet exclusion was performed on cells gated by SSC-H versus SSC-W. Viable cells were identified by FSC-A and DAPI exclusion. Finally, GFP positivity was assessed by fluorescence in the FITC channel. For human ESCs, gating was performed from left to right as shown. First, cells were separated from debris by FSC-A versus SSC-A. Then, doublet exclusion was performed on cells gated by FSC-H versus FSC-W. Then, doublet exclusion was performed on cells gated by SSC-H versus SSC-W. Viable cells were identified by FSC-A and DAPI exclusion.

### 5. DETAILED DESCRIPTION

The present disclosure provides highly efficient, non-invasive, and reversible methods for selectively enriching pluripotent cells (e.g., human pluripotent cells and mouse pluripotent cells) in a heterogenous cell population using a glutamine-deficient medium. It relates to the discovery that cells with weak pluripotency-associated transcription networks are highly glutamine dependent and rapidly die in the absence of exogenous glutamine supplementation. The presently disclosed methods have the advantageous of efficiently enriching pluripotent cells in a heterogenous cell population without altering the biological properties of any individual cells. In certain embodiments, the enriched pluripotent cells are self-renewing pluripotent cells. In certain embodiments, the pluripotent cells are enriched from an embryonic stem cell population that has been passaged in vitro, and thus contains both pluripotent and non-pluripotent cells. In certain embodiments, the pluripotent cells are fully reprogrammed pluripotent cells. In certain embodiments, the fully reprogrammed pluripotent cells are selected from a heterogenous cell population derived from somatic cells that have been subject to reprogramming to induce acquired pluripotency, where the heterogenous cell population contains fully reprogrammed pluripotent cells and not fully reprogrammed cells. In certain embodiments, the fully reprogrammed pluripotent cells are fully reprogrammed induced pluripotent cells.

The present disclosure also relates to compositions comprising pluripotent cells (e.g., self-renewing pluripotent cells) and fully reprogrammed pluripotent cells enriched in accordance to the methods disclosed herein. The present disclosure further relates to kits for selectively enriching pluripotent cells (e.g., self-renewing pluripotent cells) and fully reprogrammed pluripotent cells.

Non-limiting embodiments of the invention are described by the present specification and Examples.

For purposes of clarity of disclosure and not by way of limitation, the detailed description is divided into the following subsections:

5.1 Definitions;

5.2 Methods for enriching pluripotent cells;

5.3 Compositions comprising pluripotent cells; and

5.4 Kits.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “in vitro” refers to an artificial environment and to processes or reactions that occur within an artificial environment. In vitro environments exemplified, but are not limited to, test tubes and cell cultures.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of pluripotent cells. Alternatively, the population may comprise more than one cell type, for example a mixed cell population, e.g., a mixed population of pluripotent and non-pluripotent cells.

As used herein, the term “stem cell” refers to a cell with the ability to divide for indefinite periods in culture and to give rise to specialized cells. A human stem cell refers to a stem cell that is from a human. A mouse stem cell refers to a stem cell that is from a mouse.

As used herein, the term “embryonic stem cell line” refers to a population of embryonic stem cells which have been cultured under in vitro conditions that allow proliferation without differentiation for up to days, months to years.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like. Non-limiting exemplary somatic cells that can be reprogrammed into iPS cells include keratinocytes, fibroblasts, hepatocytes, and gastric epithelial cells.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “cell culture” refers to a growth of cells in vitro in an artificial medium for research or medical treatment.

As used herein, the term “medium” or “culture medium” interchangeably refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “expressing” in relation to a gene or protein refers to making an mRNA or protein which can be observed using assays such as microarray assays, antibody staining assays, and the like.

### 5.2 Methods for Enriching Pluripotent Cells

The present disclosure provides methods for selectively enriching pluripotent cells in a cell population, where the cell population is a mixed cell population comprising pluripotent and non-pluripotent cells. In certain embodiments, the cell population is a stem cell population that has been passaged in vitro for at least once. When passaged in vitro, cells within the stem cell population may lose their pluripotency and/or self-renewal potential, and differentiate into non-pluripotent cells, thus results in a heterogenous cell population that contains both pluripotent and non-pluripotent cells.

The presently disclosed methods include culturing the cell population in a glutamine-deficient medium, and thus selectively enriching pluripotent cells in the cell population. Non-limiting examples of stem cells include embryonic stem cells (ESC), induced pluripotent stem cells (iPSC), parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell population is a embryonic stem cell population. In certain embodiments, the cell population is a human or a mouse cell population. In certain embodiments, the cell population is a human stem cell or a mouse stem cell population.

The present disclosure also provides methods for selectively enriching fully reprogrammed pluripotent cells in a cell population, where the cell population is a mixed cell population comprising fully reprogrammed pluripotent cells and not fully reprogrammed cells. In certain embodiments, the fully programmed pluripotent cells are fully programmed induced pluripotent cells. In certain embodiments, the cell population is derived from somatic cells that have been subject to nuclear reprogramming in order to induce the somatic cells to reacquire pluripotency. Reprogramming of somatic cells is an inefficient process with low efficacy and persistence of incomplete reprogrammed cells. During the process of such reprogramming, some somatic cells are fully reprogrammed such that they fully acquired pluripotency and have the capacity for multi-linage differentiation, e.g., giving rise to all three germ layers in vivo. However, some somatic cells are not fully reprogrammed, i.e., not reprogrammed or only partially reprogrammed, and do not fully acquired pluripotency. These cells do not have the capacity for multilineage differentiation. The presently disclosed methods improve reprogramming efficiency by culturing the cell population comprising fully reprogrammed pluripotent cells and not fully reprogrammed cells in a glutamine deficient medium, and thus selectively enriched the fully reprogrammed pluripotent cells in the cell population. In certain embodiments, the cell population is a human or a mouse cell population. In certain embodiments, the cell population is derived from somatic cells that have been subject to reprogramming to induce pluripotency. In certain embodiments, the somatic cells are selected from the group consisting of keratinocytes, fibroblasts, hepatocytes, gastric epithelial cells, endothelial cells, B cells, peripheral blood mononuclear cells, and combinations thereof. In certain embodiments, the somatic cells are fibroblasts.

In certain embodiments, the presently disclosed methods comprise culturing the cell population in a glutamine-deficient medium (i.e., glutamine-free medium). Any suitable glutamine-free media known in the art can be used with the presently disclosed methods. Non-limiting exemplary glutamine-free media include glutamine-free Dulbecco's Modified Eagle Medium (DMEM) media, glutamine-free Neurobasal media, glutamine-free Knockout® Serum Replacement (“KSR”) media, glutamine-free N2 media, glutamine-free Essential 8®/Essential 6® (“E8/E6”) media, glutamine-free DMEM:F12 media, glutamine-free F12 media, glutamine-free RPMI media, glutamine-free Leibovitz's L-15 media, glutamine-free Eagle's Minimum Essential media, glutamine-free McCoy's 5 A media, and glutamine-free F-12K media.

In certain embodiments, the presently disclosed methods comprise culturing the cell population in a glutamine-deficient medium transiently. In certain embodiments, the presently disclosed methods comprise culturing the cell population in a glutamine-deficient medium for at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, or at least about 48 hours. In certain embodiments, the presently disclosed methods comprise culturing the cell population in a glutamine-deficient medium for at least about 8 hours. In certain embodiments, the presently disclosed methods comprise culturing the cell population in a glutamine-deficient medium for at least about 23 hours, at least about 24 hours, or at least about 25 hours.

In certain embodiments, the presently disclosed methods comprise culturing the cell population in a glutamine-deficient medium for at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 25 hours, at most about 26 hours, at most about 27 hours, at most about 28 hours, at most about 29 hours, at most about 30 hours, at most about 31 hours, at most about 32 hours, at most about 33 hours, at most about 34 hours, at most about 35 hours, at most about 36 hours, at most about 37 hours, at most about 38 hours, at most about 39 hours, at most about 40 hours, at most about 41 hours, at most about 42 hours, at most about 43 hours, at most about 44 hours, at most about 45 hours, at most about 46 hours, at most about 47 hours, or at most about 48 hours. In certain embodiments, the presently disclosed methods comprise culturing the cell population in a glutamine-deficient medium for at most about 8 hours. In certain embodiments, the presently disclosed methods comprise culturing the cell population in a glutamine-deficient medium for at most about 23 hours, at most about 24 hours, or at most about 25 hours.

In certain embodiments, the presently disclosed methods comprise culturing the cell population in a glutamine-deficient medium for between about 4 hours and about 10 hours, between about 4 hours and about 8 hours, between about 4 hours and about 6 hours, between about 7 hours and about 9 hours, between about 8 hours and about 48 hours, between about 8 hours and about 44 hours, between about 8 hours and about 40 hours, between about 8 hours and about 36 hours, between about 8 hours and about 32 hours, between about 8 hours and about 28 hours, between about 8 hours and about 24 hours, between about 8 hours and about 20 hours, between about 8 hours and about 16 hours, between about 8 hours and about 12 hours, between about 10 hours and about 48 hours, between about 10 hours and about 44 hours, between about 10 hours and about 40 hours, between about 10 hours and about 36 hours, between about 10 hours and about 32 hours, between about 10 hours and about 28 hours, between about 10 hours and about 24 hours, between about 10 hours and about 20 hours, between about 10 hours and about 16 hours, between about 10 hours and about 12 hours, between about 12 hours and about 48 hours, between about 12 hours and about 44 hours, between about 12 hours and about 40 hours, between about 12 hours and about 36 hours, between about 12 hours and about 32 hours, between about 12 hours and about 28 hours, between about 12 hours and about 24 hours, between about 12 hours and about 20 hours, between about 12 hours and about 16 hours, between about 14 hours and about 48 hours, between about 14 hours and about 44 hours, between about 14 hours and about 40 hours, between about 14 hours and about 36 hours, between about 14 hours and about 32 hours, between about 14 hours and about 28 hours, between about 14 hours and about 24 hours, between about 14 hours and about 20 hours, between about 14 hours and about 16 hours, between about 16 hours and about 48 hours, between about 16 hours and about 44 hours, between about 16 hours and about 40 hours, between about 16 hours and about 36 hours, between about 16 hours and about 32 hours, between about 16 hours and about 28 hours, between about 16 hours and about 24 hours, between about 16 hours and about 20 hours, between about 18 hours and about 48 hours, between about 18 hours and about 44 hours, between about 18 hours and about 40 hours, between about 18 hours and about 36 hours, between about 18 hours and about 32 hours, between about 18 hours and about 28 hours, between about 18 hours and about 24 hours, between about 18 hours and about 20 hours, between about 20 hours and about 48 hours, between about 20 hours and about 44 hours, between about 20 hours and about 40 hours, between about 20 hours and about 36 hours, between about 20 hours and about 32 hours, between about 20 hours and about 28 hours, between about 20 hours and about 24 hours, between about 22 hours and about 48 hours, between about 22 hours and about 44 hours, between about 22 hours and about 40 hours, between about 22 hours and about 36 hours, between about 22 hours and about 32 hours, between about 22 hours and about 28 hours, between about 22 hours and about 24 hours, between about 24 hours and about 48 hours, between about 24 hours and about 44 hours, between about 24 hours and about 40 hours, between about 24 hours and about 36 hours, between about 24 hours and about 32 hours, between about 24 hours and about 28 hours, between about 26 hours and about 48 hours, between about 26 hours and about 44 hours, between about 26 hours and about 40 hours, between about 26 hours and about 36 hours, between about 26 hours and about 32 hours, between about 26 hours and about 28 hours, between about 28 hours and about 48 hours, between about 28 hours and about 44 hours, between about 28 hours and about 40 hours, between about 28 hours and about 36 hours, between about 28 hours and about 32 hours, between about 30 hours and about 48 hours, between about 30 hours and about 44 hours, between about 30 hours and about 40 hours, between about 30 hours and about 36 hours, between about 30 hours and about 32 hours, between about 32 hours and about 48 hours, between about 32 hours and about 44 hours, between about 32 hours and about 40 hours, between about 32 hours and about 36 hours, between about 34 hours and about 48 hours, between about 34 hours and about 44 hours, between about 34 hours and about 40 hours, between about 34 hours and about 36 hours, between about 36 hours and about 48 hours, between about 36 hours and about 44 hours, between about 36 hours and about 40 hours, between about 38 hours and about 48 hours, between about 38 hours and about 44 hours, between about 38 hours and about 40 hours, between about 40 hours and about 48 hours, between about 40 hours and about 44 hours, or between about 44 hours and about 48 hours. In certain embodiments, the presently disclosed methods comprise culturing the cell population in a glutamine-deficient medium for between about 22 hours and about 26 hours, or between 23 hours and 25 hours.

In certain embodiments, the presently disclosed methods comprise culturing the cell population in a glutamine-deficient medium for about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours. In certain embodiments, the presently disclosed methods comprise culturing the cell population in a glutamine-deficient medium for about 8 hours. In certain embodiments, the presently disclosed methods comprise culturing the cell population in a glutamine-deficient medium for about 23 hours, about 24 hours, or about 25 hours.

In certain embodiments, the presently disclosed methods further comprise culturing the cell population in a complete medium before culturing the cell population in the glutamine deficient medium, where the complete medium comprises glutamine. The cell population can be cultured in the complete medium indefinitely before culturing the cell population in the glutamine deficient medium. In certain embodiments, before culturing the cell population in the glutamine deficient medium, the cell population is cultured in the complete medium for at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, or at least about 48 hours. In certain embodiments, before culturing the cell population in the glutamine deficient medium, the cell population is cultured in the complete medium for at least about 24 hours.

In certain embodiments, before culturing the cell population in the glutamine deficient medium, the cell population is cultured in the complete medium about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours or more.

In certain embodiments, the presently disclosed methods further comprise culturing the cell population in a complete medium after culturing the cell population in the glutamine deficient medium, where the complete medium comprises glutamine. In certain embodiments, after culturing the cell population in the glutamine deficient medium, the cell population is cultured in the complete medium for at least about 20 hours, at least about 24 hours, at least about 28 hours, at least about 32 hours, at least about 36 hours, at least about 40 hours, at least about 44 hours, at least about 48 hours, at least about 60 hours, at least about 72 hours, at least about 4 days, at least about 5 days, at least about 6 days, at least about 7 days, at least about 8 days, at least about 9 days, at least about 10 days, at least about 15 days, or at least about 20 days. In certain embodiments, after culturing the cell population in the glutamine deficient medium, the cell population is cultured in the complete medium for at least about 24 hours, or at least about 48 hours.

In certain embodiments, after culturing the cell population in the glutamine deficient medium, the cell population is cultured in the complete medium for at most about 20 hours, at most about 24 hours, at most about 28 hours, at most about 32 hours, at most about 36 hours, at most about 40 hours, at most about 44 hours, at most about 48 hours, at most about 60 hours, at most about 72 hours, at most about 4 days, at most about 5 days, at most about 6 days, at most about 7 days, at most about 8 days, at most about 9 days, at most about 10 days, at most about 15 days, or at most about 20 days. In certain embodiments, after culturing the cell population in the glutamine deficient medium, the cell population is cultured in the complete medium for at most about 48 hours, or at most about 8 days.

In certain embodiments, after culturing the cell population in the glutamine deficient medium, the cell population is cultured in the complete medium for between about 20 hours and about 20 days, between about 20 hours and about 15 days, between about 20 hours and about 10 days, between about 20 hours and about 8 days, between about 20 hours and about 6 days, between about 20 hours and about 5 days, between about 20 hours and about 4 days, between about 20 hours and about 72 hours, between about 20 hours and about 60 hours, between about 20 hours and about 48 hours, between about 20 hours and about 44 hours, between about 20 hours and about 40 hours, between about 20 hours and about 36 hours, between about 20 hours and about 32 hours, between about 20 hours and about 28 hours, between about 20 hours and about 24 hours, between about 24 hours and about 20 days, between about 24 hours and about 15 days, between about 24 hours and about 10 days, between about 24 hours and about 8 days, between about 24 hours and about 6 days, between about 24 hours and about 5 days, between about 24 hours and about 4 days, between about 24 hours and about 72 hours, between about 24 hours and about 60 hours, between about 24 hours and about 48 hours, between about 24 hours and about 44 hours, between about 24 hours and about 40 hours, between about 24 hours and about 36 hours, between about 24 hours and about 32 hours, between about 24 hours and about 28 hours, between about 28 hours and about 20 days, between about 28 hours and about 15 days, between about 28 hours and about 10 days, between about 28 hours and about 8 days, between about 28 hours and about 6 days, between about 28 hours and about 5 days, between about 28 hours and about 4 days, between about 28 hours and about 72 hours, between about 28 hours and about 60 hours, between about 28 hours and about 48 hours, between about 28 hours and about 44 hours, between about 28 hours and about 40 hours, between about 28 hours and about 36 hours, between about 28 hours and about 32 hours, between about 32 hours and about 20 days, between about 32 hours and about 15 days, between about 32 hours and about 10 days, between about 32 hours and about 8 days, between about 32 hours and about 6 days, between about 32 hours and about 5 days, between about 32 hours and about 4 days, between about 32 hours and about 72 hours, between about 32 hours and about 60 hours, between about 32 hours and about 48 hours, between about 32 hours and about 44 hours, between about 32 hours and about 40 hours, between about 32 hours and about 36 hours, between about 36 hours and about 20 days, between about 36 hours and about 15 days, between about 36 hours and about 10 days, between about 36 and about 8 days, between about 36 hours and about 6 days, between about 36 hours and about 5 days, between about 36 hours and about 4 days, between about 36 hours and about 72 hours, between about 36 hours and about 60 hours, between about 36 hours and about 48 hours, between about 36 hours and about 44 hours, between about 36 hours and about 40 hours, between about 60 hours and about 20 days, between about 60 hours and about 15 days, between about 60 hours and about 10 days, between about 60 hours and about 8 days, between about 60 hours and about 6 days, between about 60 hours and about 5 days, between about 60 hours and about 4 days, between about 60 hours and about 72 hours, between about 3 days and about 20 days, between about 3 days and about 15 days, between about 3 days and about 10 days, between about 3 days and about 8 days, between about 3 days and about 6 days, between about 3 days and about 5 days, between about 3 days and about 4 days, between about 4 days and about 20 days, between about 4 days and about 15 days, between about 4 days and about 10 days, between about 4 days and about 8 days, between about 4 days and about 6 days, between about 4 days and about 5 days, between about 5 days and about 20 days, between about 5 days and about 15 days, between about 5 days and about 10 days, between about 5 days and about 8 days, between about 5 days and about 6 days, between about 5 days and about 7 days, between about 6 days and about 20 days, between about 6 days and about 15 days, between about 6 days and about 10 days, between about 6 days and about 8 days, between about 6 days and about 7 days, between about 7 days and about 8 days, or between about 7 days and 9 days. In certain embodiments, after culturing the cell population in the glutamine deficient medium, the cell population is cultured in the complete medium for between about 46 hours and about 50 hours.

In certain embodiments, after culturing the cell population in the glutamine deficient medium, the cell population is cultured in the complete medium for about 20 hours, about 24 hours, about 28 hours, about 32 hours, about 36 hours, about 40 hours, about 44 hours, about 48 hours, about 60 hours, about 72 hours, about 4 days, about 5 days, about 6 days, about 8 days, about 9 days, about 10 days, about 15 days, or about 20 days. In certain embodiments, after culturing the cell population in the glutamine deficient medium, the cell population is cultured in the complete medium for about 46 hours, about 48 hours, about 48 hours, about 49 hours, or about 50 hours.

In certain embodiments, the presently disclosed methods comprise culturing the cell population in a glutamine-deficient medium for about 24 hours, then culturing the cell population in a complete medium comprising glutamine for about 48 hours. In certain embodiments, the presently disclosed methods comprise culturing the cell population in a complete medium comprising glutamine for at least about 24 hours, then culturing the cell population in a glutamine-deficient medium for about 24 hours. In certain embodiments, the presently disclosed methods comprise culturing the cell population in a first complete medium comprising glutamine for at least about 24 hours, then culturing the cell population in a glutamine-deficient medium for about 24 hours, then culturing the cell population in second complete medium for about 48 hours. Any suitable glutamine-containing medium known in the art can be used as the complete medium with the presently disclosed methods.

The presently disclosed methods are highly efficient in enriching the desired pluripotent cells (e.g., self-renewing pluripotent cells, fully reprogrammed pluripotent cells) in the cell population, such that the pluripotent cells are enriched in the cell population at a very high level. Reprogramming can be a low efficient process, where the fully reprogrammed pluripotent cells can be at a level of as low as about 0.1% of the cell population before the cell population is subject to the enrichment methods disclosed herein. In certain embodiments, the methods disclosed herein, therefore, enrich the fully reprogrammed cells in the cell population to a level, even though low, is relatively high as compared to the level in the cell population that has not been subject to the enrichment methods disclosed herein.

In certain embodiments, the pluripotent (e.g., self-renewing pluripotent cells, fully reprogrammed pluripotent cells) are enriched in the cell population at a level of at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99%, at most about 0.5%, at most about 1%, at most about 2%, at most about 3%, at most about 4%, at most about 5%, at most about 6%, at most about 7%, at most about 8%, at most about 9%, at most about 10%, at most about 15%, at most about 20%, at most about 25%, at most about 30%, at most about 35%, at most about 40%, at most about 45%, at most about 50%, at most about 55%, at most about 55%, at most about 60%, at most about 65%, at most about 70%, at most about 75%, at most about 80%, at most about 85%, at most about 90%, at most about 95%, at most about 99%, or at most about 100%.

In certain embodiments, the pluripotent (e.g., self-renewing pluripotent cells, fully reprogrammed pluripotent cells) are enriched in the cell population at a level of between about 0.5% and about 1%, between about 0.5% and about 2%, between about 1% and about 10%, between about 1% and about 5%, between about 2% and about 8%, between about 2% and about 4%, between about 4% and about 6%, between about 4% and about 8%, between about 5% and about 10%, between about 6% and about 8%, between about 8% and about 10%, between about 10% and about 100%, between about 10% and about 90%, between about 10% and about 80%, between about 10% and about 70%, between about 10% and about 60%, between about 10% and about 50%, between about 10% and about 40%, between about 10% and about 30%, between about 10% and about 20%, between about 10% and about 15%, between about 20% and about 100%, between about 20% and about 90%, between about 20% and about 80%, between about 20% and about 70%, between about 20% and about 60%, between about 20% and about 50%, between about 20% and about 40%, between about 20% and about 30%, between about 20% and about 25%, between about 30% and about 100%, between about 30% and about 90%, between about 30% and about 80%, between about 30% and about 70%, between about 30% and about 60%, between about 30% and about 50%, between about 30% and about 40%, between about 30% and about 35%, between about 40% and about 100%, between about 40% and about 90%, between about 40% and about 80%, between about 40% and about 70%, between about 40% and about 60%, between about 40% and about 50%, between about 40% and about 45%, between about 50% and about 100%, between about 50% and about 99%, between about 50% and about 95%, between about 50% and about 90%, between about 50% and about 85%, between about 50% and about 80%, between about 50% and about 75%, between about 50% and about 70%, between about 50% and about 65%, between about 50% and about 60%, between about 50% and about 55%, between about 55% and about 100%, between about 55% and about 99%, between about 55% and about 95%, between about 55% and about 90%, between about 55% and about 85%, between about 55% and about 80%, between about 55% and about 75%, between about 55% and about 70%, between about 55% and about 65%, between about 55% and about 60%, between about 60% and about 100%, between about 60% and about 99%, between about 60% and about 95%, between about 60% and about 90%, between about 60% and about 85%, between about 60% and about 80%, between about 60% and about 75%, between about 60% and about 70%, between about 60% and about 65%, between about 65% and about 100%, between about 65% and about 99%, between about 65% and about 95%, between about 65% and about 90%, between about 65% and about 85%, between about 65% and about 80%, between about 65% and about 75%, between about 65% and about 70%, between about 70% and about 100%, between about 70% and about 99%, between about 70% and about 95%, between about 70% and about 90%, between about 70% and about 85%, between about 70% and about 80%, between about 70% and about 75%, between about 70% and about 100%, between about 70% and about 99%, between about 70% and about 95%, between about 70% and about 90%, between about 70% and about 85%, between about 70% and about 80%, between about 70% and about 75%, between about 75% and about 100%, between about 75% and about 99%, between about 75% and about 95%, between about 75% and about 90%, between about 75% and about 85%, between about 75% and about 80%, between about 80% and about 100%, between about 80% and about 99%, between about 80% and about 95%, between about 80% and about 90%, between about 80% and about 85%, between about 85% and about 100%, between about 85% and about 99%, between about 85% and about 95%, between about 85% and about 90%, between about 90% and about 100%, between about 95% and about 99%, between about 95% and about 100%, or between about 99% and about 100%.

In certain embodiments, the pluripotent (e.g., self-renewing pluripotent cells, fully reprogrammed pluripotent cells) are enriched in the cell population to a level of about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% of the cell population. In certain embodiments, the pluripotent (e.g., self-renewing pluripotent cells, fully reprogrammed pluripotent cells) are enriched in the cell population to a level of about 90%, about 95, about 99%, or about 100% of the cell population.

In certain embodiments, the methods disclosed herein selectively increase the relative level of the desired pluripotent cells (e.g., self-renewing pluripotent cells, fully reprogrammed pluripotent cells) in the cell population as compared to the level of pluripotent cells (e.g., self-renewing pluripotent cells, fully reprogrammed pluripotent cells) in a cell population that has not been subject to the methods of enriching disclosed herein.

In certain embodiments, the methods disclosed herein selectively increase the relative level of the desired pluripotent cells (e.g., self-renewing pluripotent cells, fully reprogrammed pluripotent cells) in the cell population at least about 10%, at least about 20%, at least about 30%, at least about 40%, at least about 50%, at least about 60%, at least about 70%, at least about 80%, at least about 100%, at least about 150%, at least about 200%, at least about 250%, at least about 300%, at least about 350%, at least about 400%, at least about 450%, at least about 500%, at most about 10%, at most about 20%, at most about 30%, at most about 40%, at most about 50%, at most about 60%, at most about 70%, at most about 80%, at most about 100%, at most about 150%, at most about 200%, at most about 250%, at most about 300%, at most about 350%, at most about 400%, at most about 450%, or at most about 500% as compared to the level of pluripotent cells (e.g., self-renewing pluripotent cells, fully reprogrammed pluripotent cells) in a cell population that has not been subject to the methods of enriching disclosed herein.

In certain embodiments, the methods disclosed herein selectively increase the relative level of the desired pluripotent cells (e.g., self-renewing pluripotent cells, fully reprogrammed pluripotent cells) in the cell population between about 10% and about 500%, between about 10% and about 400%, between about 10% and about 300%, between about 10% and about 200%, between about 10% and about 100%, between about 10% and about 80%, between about 10% and about 60%, between about 10% and about 50%, between about 10% and about 40%, between about 10% and about 30%, between about 10% and about 20%, between about 20% and about 500%, between about 20% and about 400%, between about 20% and about 300%, between about 20% and about 200%, between about 20% and about 100%, between about 20% and about 80%, between about 20% and about 60%, between about 20% and about 50%, between about 20% and about 40%, between about 20% and about 30%, between about 40% and about 500%, between about 40% and about 400%, between about 40% and about 300%, between about 40% and about 200%, between about 40% and about 100%, between about 40% and about 80%, between about 40% and about 60%, between about 40% and about 50%, between about 50% and about 500%, between about 50% and about 400%, between about 50% and about 300%, between about 50% and about 200%, between about 50% and about 100%, between about 50% and about 80%, between about 50% and about 70%, between about 50% and about 60%, between about 60% and about 500%, between about 60% and about 400%, between about 60% and about 300%, between about 60% and about 200%, between about 60% and about 100%, between about 60% and about 80%, between about 60% and about 70%, between about 80% and about 500%, between about 80% and about 400%, between about 80% and about 300%, between about 80% and about 200%, between about 80% and about 100%, between about 100% and about 500%, between about 100% and about 450%, between about 100% and about 400%, between about 100% and about 350%, between about 100% and about 300%, between about 100% and about 250%, between about 100% and about 200%, between about 100% and about 150%, between about 150% and about 500%, between about 150% and about 450%, between about 150% and about 400%, between about 150% and about 350%, between about 150% and about 300%, between about 150% and about 250%, between about 150% and about 200%, between about 200% and about 500%, between about 200% and about 450%, between about 200% and about 400%, between about 200% and about 350%, between about 200% and about 300%, between about 200% and about 250%, between about 250% and about 500%, between about 250% and about 450%, between about 250% and about 400%, between about 250% and about 350%, between about 250% and about 300%, between about 300% and about 500%, between about 300% and about 450%, between about 300% and about 400%, between about 300% and about 350%, between about 400% and about 500%, or between about 400% and about 450% as compared to the level of pluripotent cells (e.g., self-renewing pluripotent cells, fully reprogrammed pluripotent cells) in a cell population that has not been subject to the methods of enriching disclosed herein.

In certain embodiments, the methods disclosed herein selectively increase the relative level of the desired pluripotent cells (e.g., self-renewing pluripotent cells, fully reprogrammed pluripotent cells) in the cell population about 10%, about 20%, about 30%, about 40%, about 50%, about 60%, about 70%, about 80%, about 100%, about 150%, about 200%, about 250%, about 300%, about 350%, about 400%, about 450%, or about 500% as compared to the level of pluripotent cells (e.g., self-renewing pluripotent cells, fully reprogrammed pluripotent cells) in a cell population that has not been subject to the methods of enriching disclosed herein.

In certain embodiments, the pluripotency and/or self-renewal capacity of the cells are associated with the cells' ability to sustain intracellular α-ketoglutarate (αKG) in the absence of exogenous glutamine. As such, the pluripotent cells have an elevated intracellular αKG/succinate ratio as compared to the non-pluripotent cells. In certain embodiments, the self-renewing pluripotent cells have an elevated cellular αKG/succinate ratio as compared to the non-pluripotent cells. In certain embodiments, the fully reprogrammed pluripotent cells have an elevated cellular αKG/succinate ratio as compared to the not fully reprogrammed cells.

In certain embodiments, the pluripotent cells (e.g., self-renewing pluripotent cells, fully reprogrammed pluripotent cells) overexpress pluripotency-associated markers as compared to non-pluripotent cells. In certain embodiments, the pluripotent cells express a pluripotency-associated marker selected from the group consisting of Nanog, Oct4, Sox2, Esrrb, Zfp42, Klf4, Tfcp2l1, Stat3, and combinations thereof. In certain embodiments, the self-renewing pluripotent cells express a pluripotency marker selected from the group consisting of Nanog, Oct4, Sox2, Esrrb, Zfp42, Klf4, Tfcp2l1, Stat3, and combinations thereof. In certain embodiments, the fully reprogrammed pluripotent cells express a pluripotency marker selected from the group consisting of Nanog, Oct4, Sox2, Esrrb, Zfp42, Klf4, Tfcp2l1, Stat3, and combinations. In certain embodiments, the pluripotent cells express a pluripotency-associated marker selected from the group consisting of Nanog, Oct4, Sox2, and combinations thereof. In certain embodiments, the self-renewing pluripotent cells express a pluripotency marker selected from the group consisting of Nanog, Oct4, Sox2, and combinations thereof. In certain embodiments, the fully reprogrammed pluripotent cells express a pluripotency marker selected from the group consisting of Nanog, Oct4, Sox2, and combinations.

In certain embodiments, the pluripotent cells express a high level of a pluripotent marker as compared to the non-pluripotent cells, where the marker is selected from the group consisting of Nanog, Oct4, Sox2, Esrrb, Zfp42, Klf4, Tfcp2l1, Stat3, and combinations thereof. In certain embodiments, the self-renewing pluripotent cells express a high level of a pluripotent marker as compared to the non-pluripotent cells, where the marker is selected from the group consisting of Nanog, Oct4, Sox2, Esrrb, Zfp42, Klf4, Tfcp2l1, Stat3, and combinations thereof. In certain embodiments, the fully reprogrammed pluripotent cells express a high level of a pluripotent marker as compared to the not fully reprogrammed cells, where the marker is selected from the group consisting of Nanog, Oct4, Sox2, Esrrb, Zfp42, Klf4, Tfcp2l1, Stat3, and combinations thereof. In certain embodiments, the pluripotent cells express a high level of a pluripotent marker as compared to the non-pluripotent cells, where the marker is selected from the group consisting of Nanog, Oct4, Sox2, and combinations thereof. In certain embodiments, the self-renewing pluripotent cells express a high level of a pluripotent marker as compared to the non-pluripotent cells, where the marker is selected from the group consisting of Nanog, Oct4, Sox2, and combinations thereof. In certain embodiments, the fully reprogrammed pluripotent cells express a high level of a pluripotent marker as compared to the not fully reprogrammed cells, where the marker is selected from the group consisting of Nanog, Oct4, Sox2, and combinations thereof.

### 5.3 Compositions Comprising Pluripotent Cells

The present disclosure provides compositions comprising a population of pluripotent cells (e.g., self-renewing pluripotent cells, fully reprogrammed pluripotent cells) produced by the methods described herein.

In certain embodiments, the present disclosure provides compositions comprising a population of enriched pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of pluripotent cells express one or more pluripotency marker selected from the group consisting of Nanog, Oct4, Sox2, Esrrb, Zfp42, Klf4, Tfcp2l1, Stat3, and combinations thereof. In certain embodiments, the present disclosure provides compositions comprising a population of enriched self-renewing pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of self-renewing pluripotent cells express one or more pluripotency marker selected from the group consisting of Nanog, Oct4, Sox2, Esrrb, Zfp42, Klf4, Tfcp2l1, Stat3, and combinations thereof. In certain embodiments, the present disclosure provides compositions comprising a population of enriched fully reprogrammed pluripotent cells (e.g., fully reprogrammed induced pluripotent cells), wherein at least about 0.5% (e.g., at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99% or about 100%) of the population of fully reprogrammed pluripotent cells (e.g., fully reprogrammed induced pluripotent cells) express one or more pluripotency marker selected from the group consisting of Nanog, Oct4, Sox2, Esrrb, Zfp42, Klf4, Tfcp2l1, Stat3, and combinations thereof.

In certain embodiments, the present disclosure provides compositions comprising a population of enriched pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of pluripotent cells express one or more pluripotency marker selected from the group consisting of Nanog, Oct4, Sox2, and combinations thereof. In certain embodiments, the present disclosure provides compositions comprising a population of enriched self-renewing pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of self-renewing pluripotent cells express one or more pluripotency marker selected from the group consisting of Nanog, Oct4, Sox2, and combinations thereof. In certain embodiments, the present disclosure provides compositions comprising a population of enriched fully reprogrammed pluripotent cells (e.g., fully reprogrammed induced pluripotent cells), wherein at least about 0.5% (e.g., at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99%, or about 100%) of the population of fully reprogrammed pluripotent cells (e.g., fully reprogrammed induced pluripotent cells) express one or more pluripotency marker selected from the group consisting of Nanog, Oct4, Sox2, and combinations thereof.

In certain embodiments, the present disclosure provides compositions comprising a population of enriched pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of pluripotent cells express a high level of one or more pluripotency marker selected from the group consisting of Nanog, Oct4, Sox2, Esrrb, Zfp42, Klf4, Tfcp2l1, Stat3, and combinations thereof as compared to non-pluripotent cells. In certain embodiments, the present disclosure provides compositions comprising a population of enriched self-renewing pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of self-renewing pluripotent cells express a high level of one or more pluripotency marker selected from the group consisting of Nanog, Oct4, Sox2, Esrrb, Zfp42, Klf4, Tfcp2l1, Stat3, and combinations thereof as compared to non-pluripotent cells. In certain embodiments, the present disclosure provides compositions comprising a population of enriched fully reprogrammed pluripotent cells (e.g., fully reprogrammed induced pluripotent cells), wherein at least about 0.5% (e.g., at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99%, or about 100%) of the population of fully reprogrammed pluripotent cells (e.g., fully reprogrammed induced pluripotent cells) express a high level of one or more pluripotency marker selected from the group consisting of Nanog, Oct4, Sox2, Esrrb, Zfp42, Klf4, Tfcp2l1, Stat3, and combinations thereof as compared to not fully programmed cells.

In certain embodiments, the present disclosure provides compositions comprising a population of enriched pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of pluripotent cells express a high level of one or more pluripotency marker selected from the group consisting of Nanog, Oct4, Sox2, and combinations thereof as compared to non-pluripotent cells. In certain embodiments, the present disclosure provides compositions comprising a population of enriched self-renewing pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of self-renewing pluripotent cells express a high level of one or more pluripotency marker selected from the group consisting of Nanog, Oct4, Sox2, and combinations thereof as compared to non-pluripotent cells. In certain embodiments, the present disclosure provides compositions comprising a population of enriched fully reprogrammed pluripotent cells (e.g., fully reprogrammed induced pluripotent cells), wherein at least about 0.5% (e.g., at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99%, or about 100%) of the population of fully reprogrammed pluripotent cells (e.g., fully reprogrammed induced pluripotent cells) express a high level of one or more pluripotency marker selected from the group consisting of Nanog, Oct4, Sox2, and combinations thereof as compared to not fully programmed cells.

In certain embodiments, the present disclosure provides compositions comprising a population of enriched pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of pluripotent cells have an elevated cellular αKG/succinate ratio as compared to the non-pluripotent cells. In certain embodiments, the present disclosure provides compositions comprising a population of enriched self-renewing pluripotent cells, wherein at least about 90% (e.g., at least about 91%, at least about 92%, at least about 93%, at least about 94%, at least about 95%, at least about 96%, at least about 97%, at least about 98%, at least about 99%, or about 100%) of the population of self-renewing pluripotent cells have an elevated cellular αKG/succinate ratio as compared to the non-pluripotent cells. In certain embodiments, the present disclosure provides compositions comprising a population of enriched fully reprogrammed pluripotent cells (e.g., fully reprogrammed induced pluripotent cells), wherein at least about 0.5% (e.g., at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, at least about 75%, at least about 80%, at least about 85%, at least about 90%, at least about 95%, at least about 99%, or about 100%) of the population of fully reprogrammed pluripotent cells (e.g., fully reprogrammed induced pluripotent cells) have an elevated cellular αKG/succinate ratio as compared to not fully programmed cells.

In certain embodiments, the composition comprises a population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed enriched pluripotent cells (e.g., self-renewing pluripotent cells, fully reprogrammed pluripotent cells) produced by the methods described herein.

### 5.4 Kits

The present disclosure provides kits for selectively enriching pluripotent cells (e.g., self-renewing pluripotent cells). In certain embodiments, the kits comprise a glutamine-deficient medium, and a cell population comprises non-pluripotent cells and the pluripotent cells. In certain embodiments, the cell population is a stem cell population. In certain embodiments, the stem cell population has been passaged in vitro at least once. Non-limiting examples of stem cells include embryonic stem cells (ESC), induced pluripotent stem cells (iPSC), parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell population is an embryonic stem cell population. In certain embodiments, the cell population is a human or a mouse cell population. In certain embodiments, the cell population is a human stem cell or a mouse stem cell population.

The present disclosure also provides kits for selectively enriching fully reprogrammed pluripotent cells. In certain embodiments, the kits comprise a glutamine-deficient medium, and a cell population comprise not fully reprogrammed cells and the fully reprogrammed pluripotent cells. In certain embodiments, the fully programmed pluripotent cells are fully reprogrammed induced pluripotent cells. In certain embodiments, the cell population is nuclear reprogrammed somatic cells, where the reprogramming intends to induce the reacquisition of pluripotency by the somatic cells. In certain embodiments, the cell population is a human or a mouse cell population. In certain embodiments, the cell population is derived from somatic cells that have been subject to reprogramming to induce pluripotency. In certain embodiments, the somatic cells are selected from the group consisting of keratinocytes, fibroblasts, hepatocytes, gastric epithelial cells, and combinations thereof. In certain embodiments, the somatic cells are fibroblasts.

In certain embodiments, the kits further comprise instructions for selectively enriching the pluripotent cells (e.g., self-renewing pluripotent cells) or the fully reprogrammed pluripotent cells (e.g., fully reprogrammed induced pluripotent cells).

In certain embodiments, the instructions comprise culturing the cell population in the glutamine-deficient medium as described by the methods of the present disclosure (see, supra, Section 5.2).

In certain embodiments, the kits further comprise a complete medium comprising glutamine. In certain embodiments, the instructions comprise culturing the cell population in the glutamine-deficient medium as described by the methods of the present disclosure (see, supra, Section 5.2).

### 6. EXAMPLE

The presently disclosed subject matter will be better understood by reference to the following Example, which is provided as exemplary of the presently disclosed subject matter, and not by way of limitation.

### Example 1: Glutamine Independence is a Selectable Feature of Pluripotent Stem Cells

### Summary

Most rapidly proliferating mammalian cells rely on the oxidation of exogenous glutamine to support cell proliferation. It was previously found that culture of mouse embryonic stem cells in the presence of inhibitors against mitogen-activated protein kinase kinase and glycogen synthase kinase 3 beta to maintain pluripotency reduces cellular reliance on glutamine for tricarboxylic acid cycle anaplerosis, enabling embryonic stem cells to proliferate in the absence of exogenous glutamine. This Example shows that reduced dependence on exogenous glutamine is a generalizable feature of pluripotent stem cells. Enhancing self-renewal, through either overexpression of pluripotency-associated transcription factors or altered signal transduction, decreases the use of glutamine-derived carbons in the tricarboxylic acid cycle. As a result, cells with the highest potential for self-renewal can be enriched by transient culture in glutamine-deficient media. During pluripotent cell culture or reprogramming to pluripotency, transient glutamine withdrawal selectively leads to the elimination of non-pluripotent cells. These data reveal that reduced dependence on glutamine anaplerosis is an inherent feature of self-renewing pluripotent stem cells and reveal a simple, non-invasive mechanism to select for mouse and human pluripotent stem cells within a heterogeneous population during both embryonic stem cell passage and induced pluripotent cell reprogramming.

### INTRODUCTION

Given the emerging links between proliferative metabolism and cell identity, the present disclosure exploits the specific metabolic requirements of particular cell types to favor the enrichment of cells with the highest capacity for self-renewal. Mouse embryonic stem cells (ESCs) cultured under conventional conditions including serum and leukemia inhibitory factor (LIF; hereafter S/L) exhibit heterogeneous expression of key pluripotency transcription factors that denote cells with variable propensity for differentiation (Chamber et al., Nature 450, 1230-1234 (2007); Filipczyk et al., Cell Stem Cell 13, 12-13 (2013)). Addition of inhibitors against MEK and GSK3β (2i′) drive cells into a naïve “ground state” of pluripotency in which cells express relatively homogenous levels of pluripotency transcription factors and are resistant to spontaneous differentiation (Ying et al., Nature 453, 519-523 (2008)). It was previously showed that addition of 2i to mouse ESCs rewired intracellular metabolic pathways without altering proliferation rate (Carey et al., Nature 518, 413-416 (2015)). In particular, 2i-cultured ESCs decreased glutamine oxidation and increased glucose oxidation, enabling an increase in the ratio of αKG/succinate that has been mechanistically linked to the regulation of chromatin and cell identity in a variety of contexts (Carey et al., Nature 518, 413-416 (2015); Chisolm et al., Immunity 47, 251-267 e257 (2017); Liu et; al., Nat Immunol 18, 985-994 (2017); Yang et al., Cell Metab 24, 542-554 (2016)). The present disclosure resolved the issue of whether altered metabolic profiles are a specific consequence of altered signal transduction or a general feature of self-renewing ESCs. The present disclosure also discovered that both mouse and human pluripotent stem cells are able to survive and be enriched by transient culture in glutamine-deficient medium. These results demonstrate that defined metabolic profiles are an inherent feature of pluripotent stem cell identity and provide a rationale for the use of metabolic interventions as a method to manipulate heterogeneity in stem cell populations.

### Results

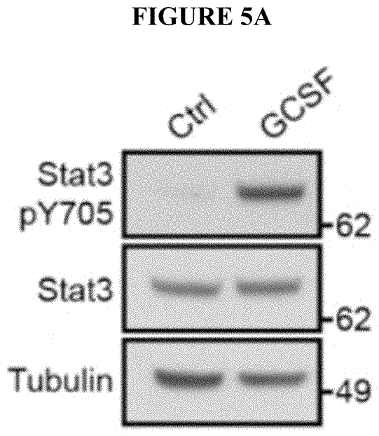

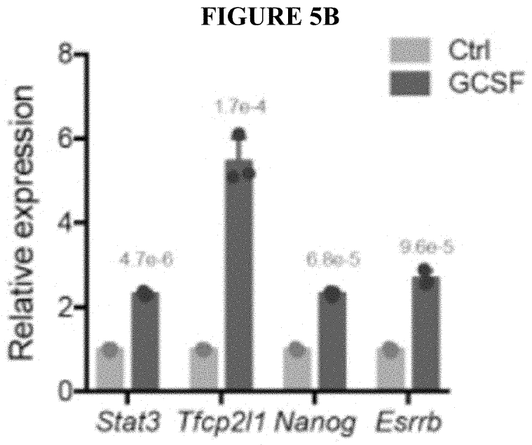

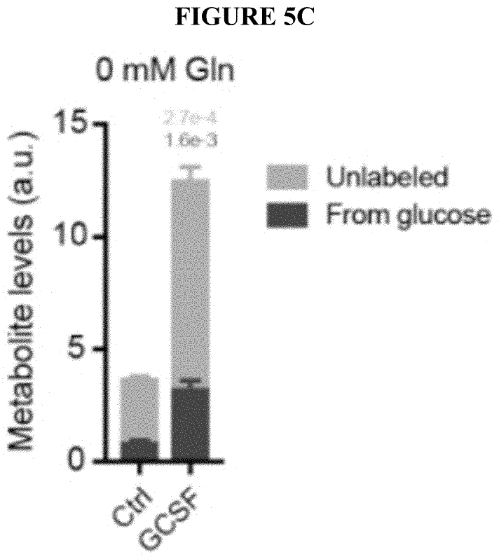

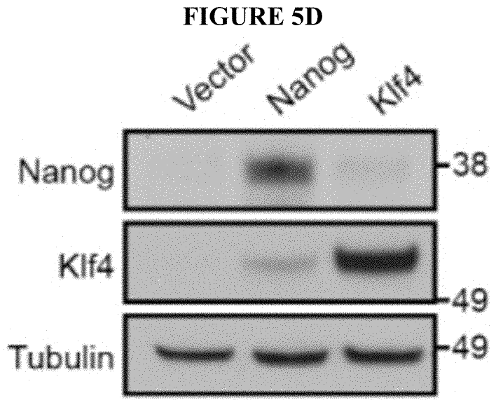

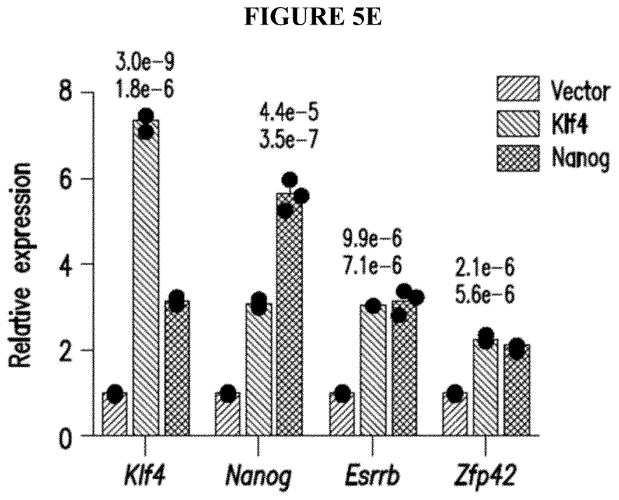

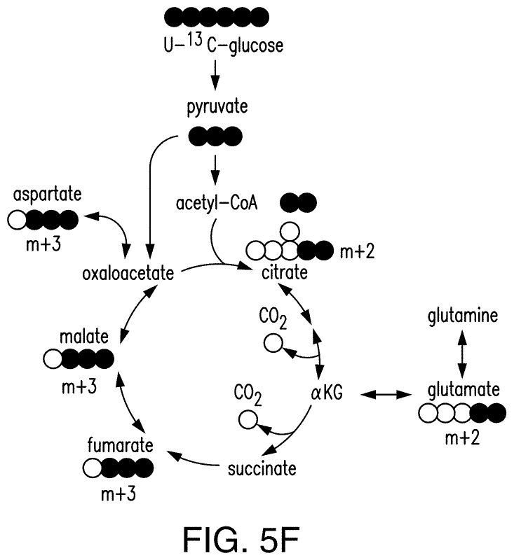

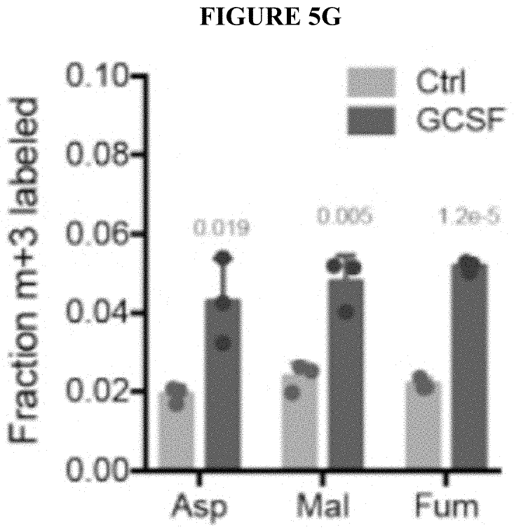

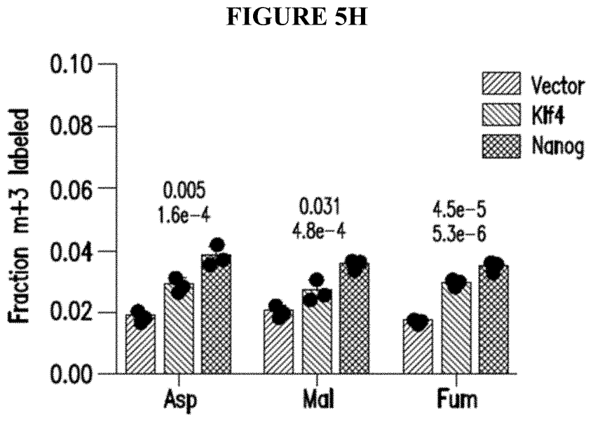

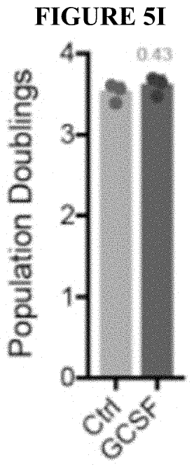

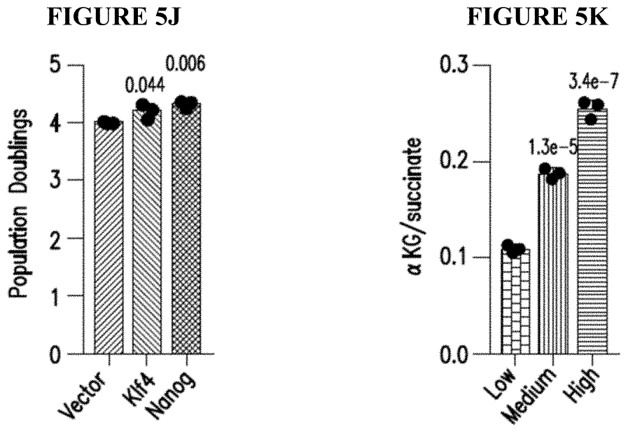

Glutamine anaplerosis is reduced in highly self-renewing ESCs. In proliferating mammalian cells in vitro, glutamine is the major source of carbon for tricarboxylic acid (TCA) cycle intermediates (DeBerardinis et al., Cell metabolism 7, 11-20 (2008)). Consequently, most cell lines including ESCs depend on exogenous glutamine for growth and proliferation (DeBerardinis et al., Cell metabolism 7, 11-20 (2008); Carey et al., Nature 518, 413-416 (2015); Tohyama et al., Cell metabolism 23, 663-674 (2016)). One notable exception is 2i-cultured mouse ESCs in the ground state of pluripotency, which can sustain proliferation in the absence of exogenous glutamine (DeBerardinis et al., Cell metabolism 7, 11-20 (2008)). It was previously found that addition of 2i reduces the contribution of glutamine-derived carbons to TCA cycle intermediates while increasing the contribution of glucose-derived carbons, thereby reducing reliance on exogenous glutamine to support TCA cycle anaplerosis (DeBerardinis et al., Cell metabolism 7, 11-20 (2008)). To determine whether the effects of 2i on mouse ESC metabolism are a specific consequence of MEK/GSK3β inhibition or a general feature of the metabolic requirements of self-renewing pluripotent stem cells, alternative methods are used to drive ESCs into the ground state of pluripotency. First, a chimeric LIF receptor was used which was engineered to respond to granulocyte colony-stimulating factor (GCSF) and harboring a mutation at tyrosine 118 to impair negative feedback by Socs3 (Burdon et al., Dev Biol 210, 30-43 (1999)). Upon treatment with GCSF, cells expressing this chimeric receptor exhibit elevated and sustained JAK/STAT3 signaling (FIG. 5A) and are stabilized in the naïve state of pluripotency regardless of the presence of differentiation-inducing stimuli (van Oosten et al., Nature communications 3, 817 (2012)).

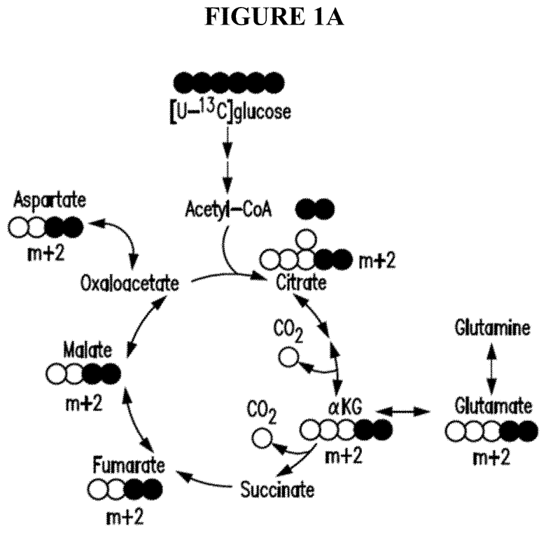

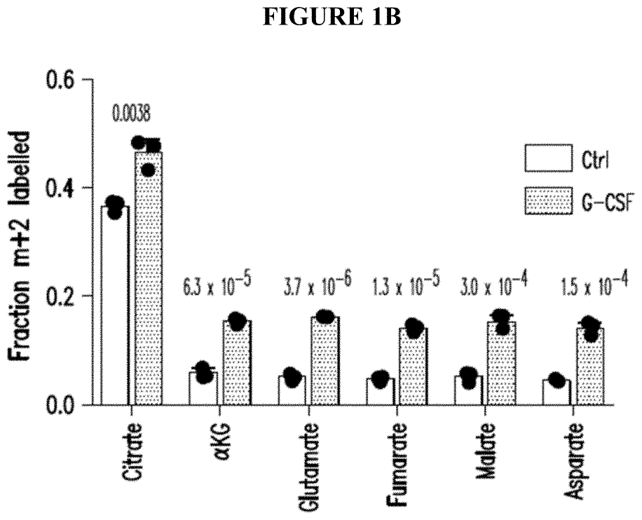

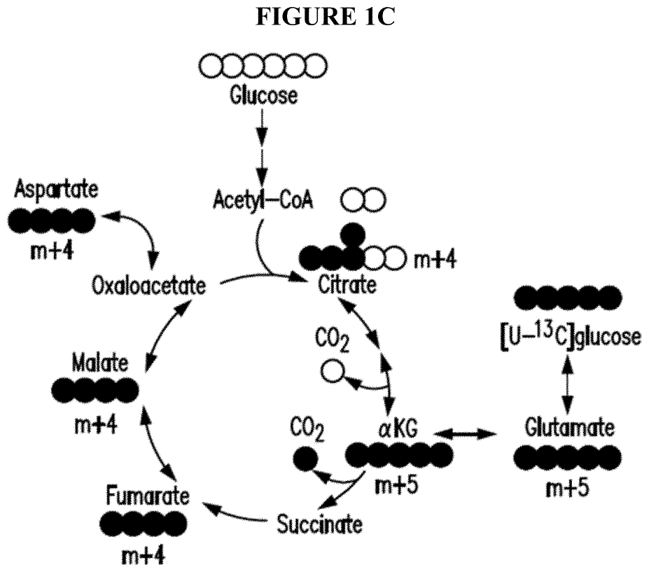

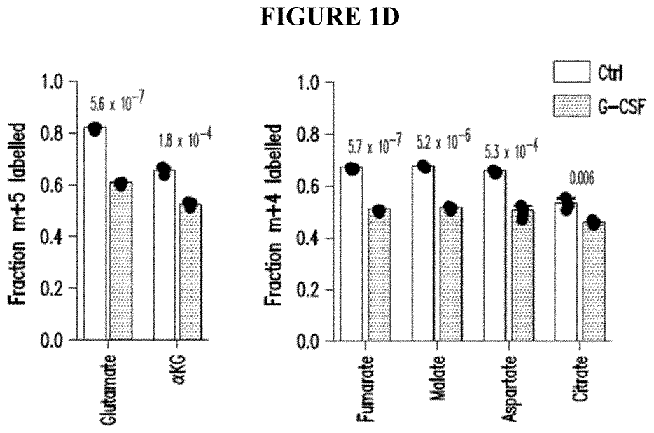

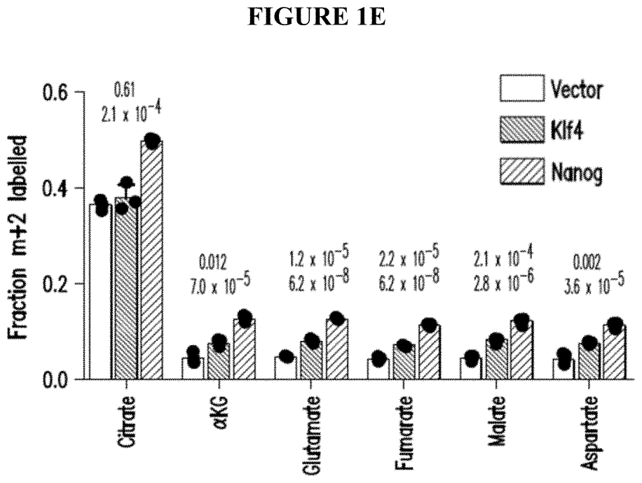

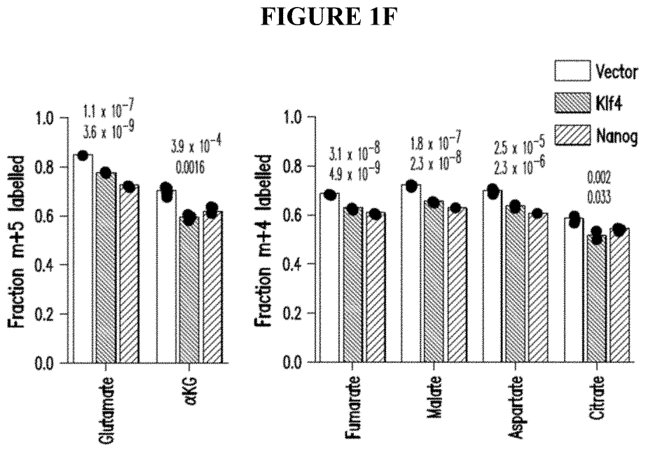

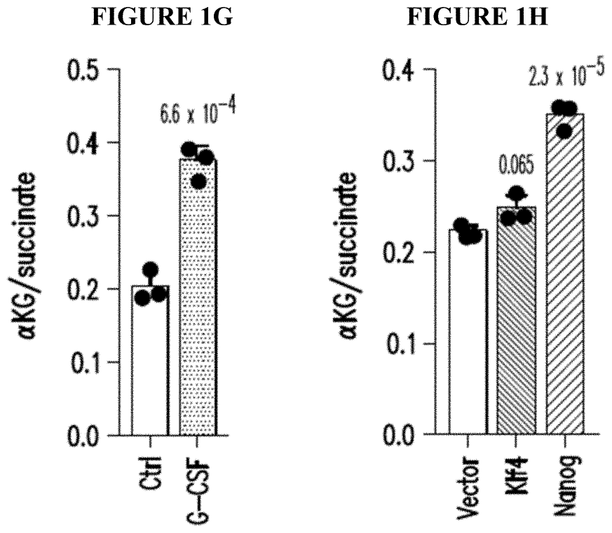

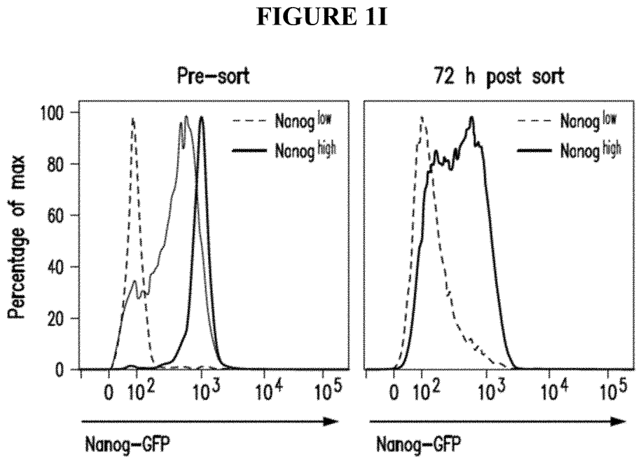

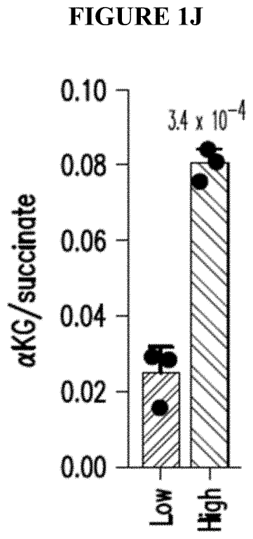

To assess glucose and glutamine utilization in these cells, gas chromatography-mass spectrometry was used to trace the fate of uniformly ¹³C-labeled glucose or glutamine. Besides enhancing expression of the Stat3 target gene Tfcp2l1 and key transcription factors associated with naïve pluripotency (Martello et al., EMBO J 32, 2561-2574 (2013)) (FIG. 5B), GCSF treatment induced metabolic alterations similar to those triggered by 2i. Specifically, GCSF increased the fraction of TCA cycle metabolites derived from oxidative decarboxylation of glucose-derived pyruvate (m+2 labeled isotopologues) and decreased the fraction of metabolites derived from glutamine catabolism (FIGS. 1A-1D). Whereas more than 80% of glutamate was derived from glutamine in control ESCs, in line with other cultured mammalian cells, GCSF reduced the fraction of glutamate derived from glutamine to 60% suggesting that GCSF-cultured ESCs can generate glutamate from sources other than glutamine (FIG. 1D). Indeed, when deprived of exogenous glutamine, GCSF-cultured ESCs were able to use glucose and other anaplerotic substrates to maintain intracellular pools of glutamate (FIG. 5C).

Both MEK/GSK3β inhibition and JAK/STAT3 activation promote naïve pluripotency by altering signal transduction. To determine whether direct activation of pluripotency gene networks is sufficient to rewire intracellular metabolic pathways, pluripotency-associated transcription factors Klf4 and Nanog were ectopically expressed (FIG. 5D). Expression of either transcription factor is sufficient to enhance self-renewal (Chambers et al., Cell 113, 643-655 (2003); Mitsui et al., Cell 113, 631-642 (2003); Zhang et al, J Biol Chem 285, 9180-9189 (2010)) and induce expression of target genes associated with naïve pluripotency (Festuccia et al., Cell stem cell 11, 477-490 (2012); Shi et al., The Journal of biological chemistry 281, 23319-23325 (2006)) (FIG. 5E). Similar to MEK/GSK3β inhibition and JAK/STAT3 activation, overexpression of Klf4 or Nanog increased the fraction of TCA cycle intermediates generated from glucose-derived carbons (FIG. 1E) while decreasing the fraction of TCA cycle intermediates derived from glutamine (FIG. 1F). Under conditions of reduced glutamine anaplerosis, pyruvate carboxylase can partially compensate to maintain TCA cycle anaplerosis (Cheng et al., Proceedings of the National Academy of Sciences of the United States of America 108, 8674-8679 (2011)). Accordingly, both JAK/STAT3-stimulated and Nanog/Klf4-expressing cells exhibited an increase in glucose-derived anaplerosis through pyruvate carboxylase (m+3 isotopologues), further underscoring the reduced reliance on glutamine anaplerosis in cells with enhanced pluripotency networks (FIGS. 5F-5H).

Together, these results suggest that interventions that enhance ESC self-renewal alter the balance of glucose and glutamine utilization in cells independently of changes in culture conditions or proliferation rates (FIGS. 5I-5J). This metabolic shift—marked by reduced glutamine anaplerosis coupled with enhanced contribution of glucose-derived carbons to TCA cycle metabolites—was previously represented as a change in the ratio of α-ketoglutarate to succinate (Carey et al., Nature 518, 413-416 (2015)). Both JAK/STAT3 induction and Nanog overexpression increase cellular αKG/succinate ratios, while Klf4, which has a more modest effect on glucose and glutamine utilization under routine culture conditions, had no notable effect on this ratio (FIGS. 1G-1H).

It was next asked whether the αKG/succinate ratio varies in correlation with the inherent self-renewal potential of ESCs. The significant population heterogeneity of ESCs cultured under conventional serum/LIF (S/L) conditions provided an opportunity to determine whether the functional heterogeneity of ESCs is accompanied by metabolic heterogeneity. To this end, the present disclosure utilized ESCs harboring a GFP reporter at the endogenous Nanog locus (Faddah et al., Cell stem cell 13, 23-29 (2013)) to sort out cells with the lowest and highest expression of Nanog (FIG. 1I, left). These Nanog-GFP cells have previously been used to illustrate that “Nanog Low” cells are more prone to differentiate than their “Nanog High” counterparts (Faddah et al., Cell stem cell 13, 23-29 (2013); Boroviak et al., Nat Cell Biol 16, 516-528 (2014)). Because of the inherent metastability of S/L-cultured ESCs, within three days of sorting both “Nanog Low” and “Nanog High” sorted populations had begun to re-establish the variable Nanog expression that characterizes S/L-cultured ESCs (FIG. 1I, right). Nevertheless, the ratio of αKG/succinate was significantly different in the two populations, with Nanog High cells characterized by an elevated αKG/succinate ratio consistent with their enhanced capacity for self-renewal (FIG. 1J). Further supporting a tight correlation between the αKG/succinate ratio and ESC self-renewal, the αKG/succinate ratio increased progressively from Nanog-low to Nanog-intermediate and Nanog-high populations (FIG. 5K).

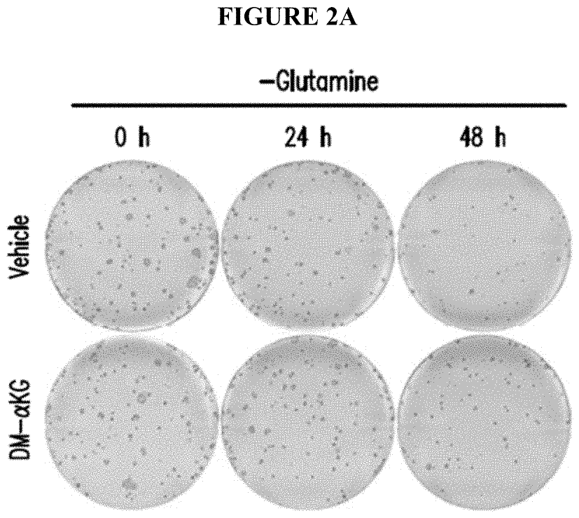

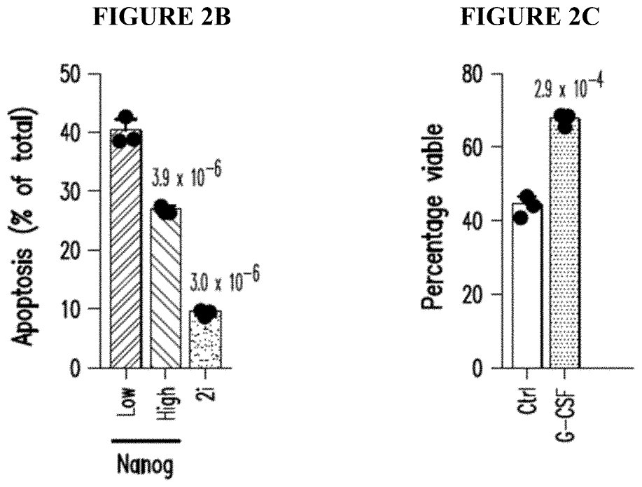

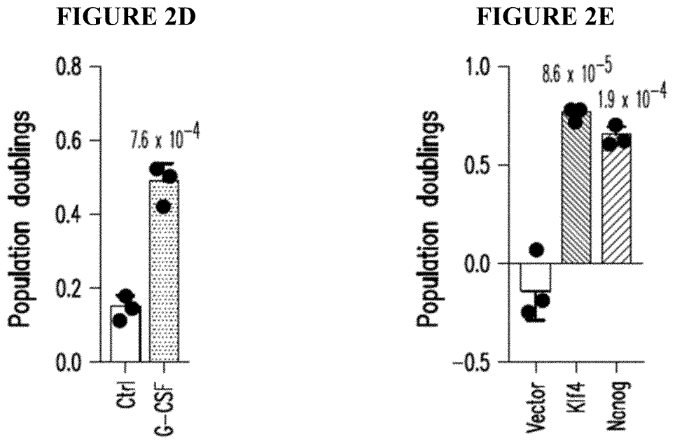

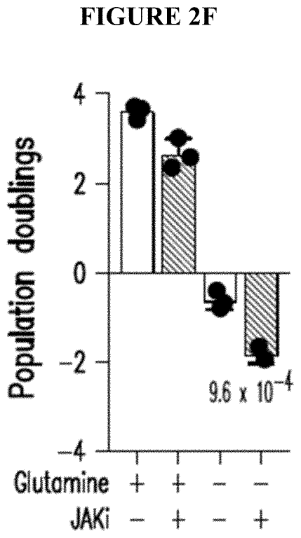

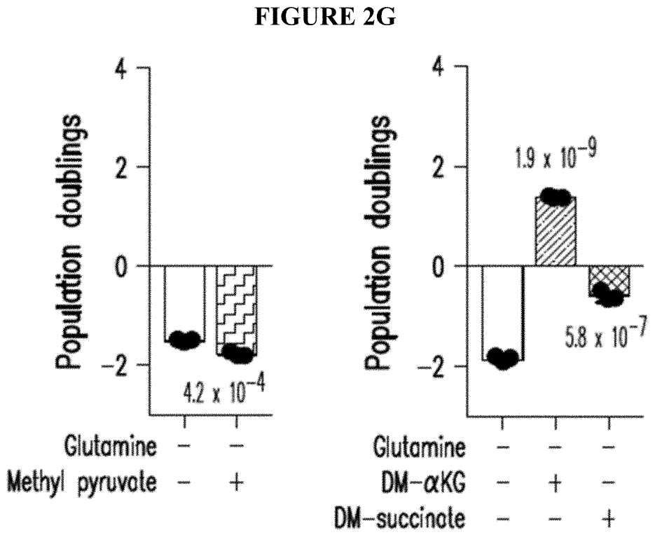

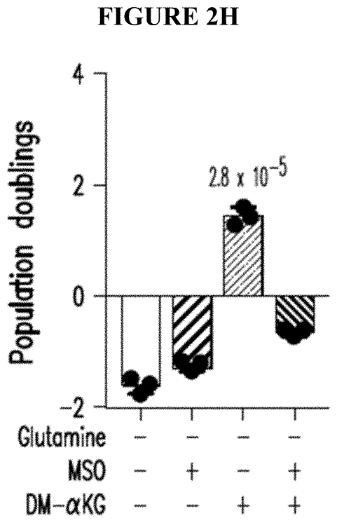

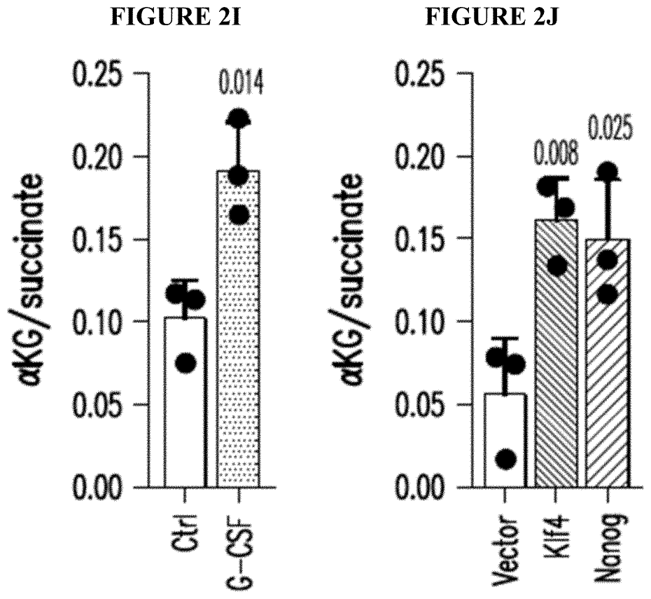

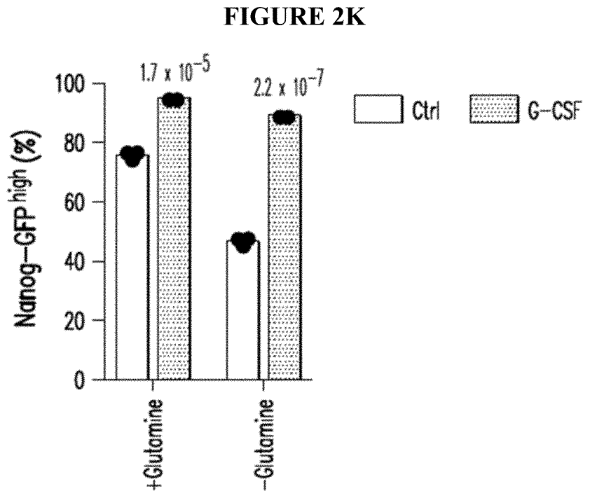

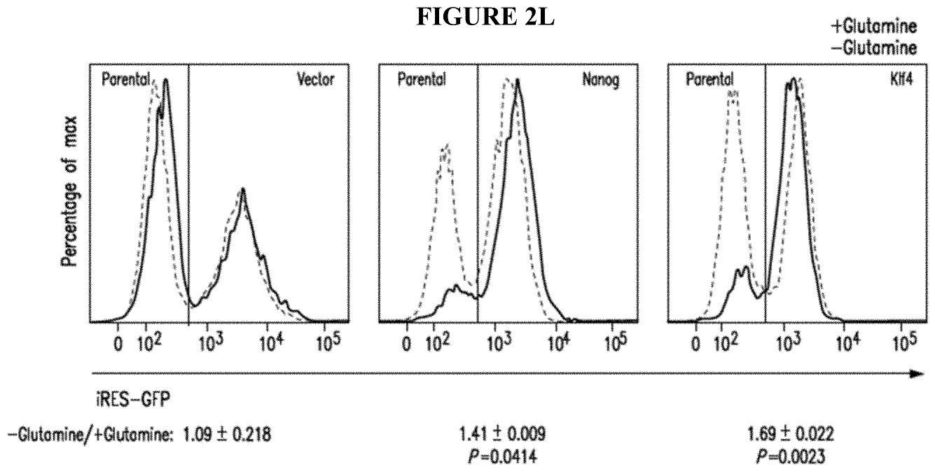

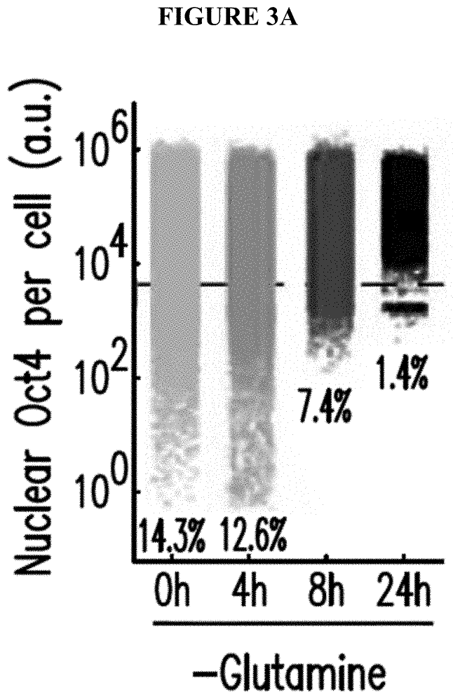

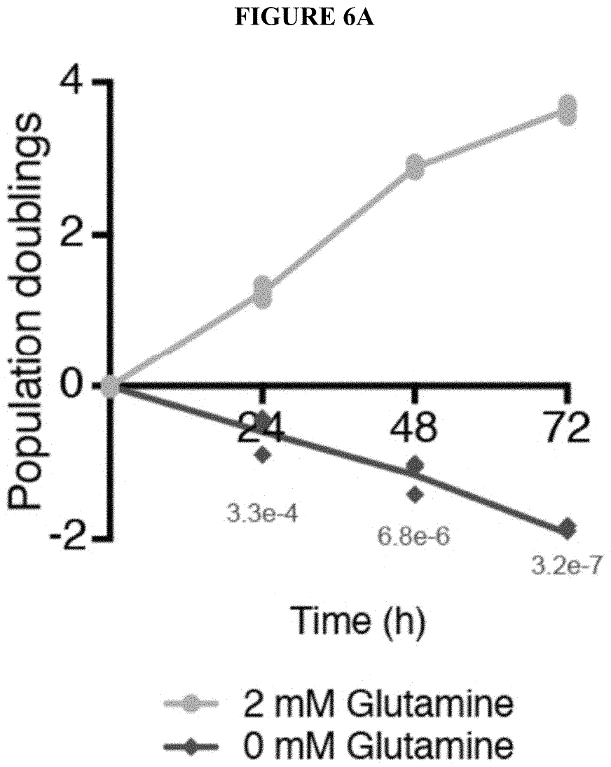

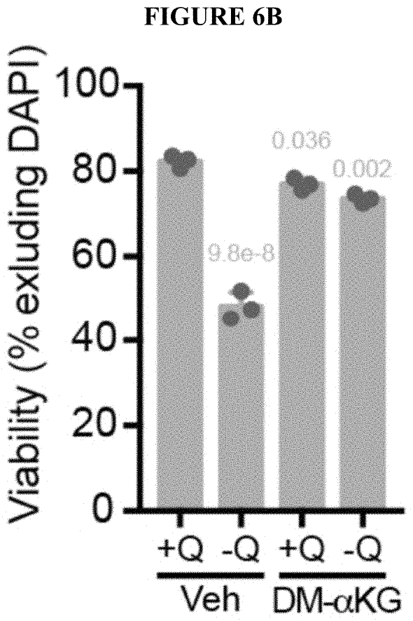

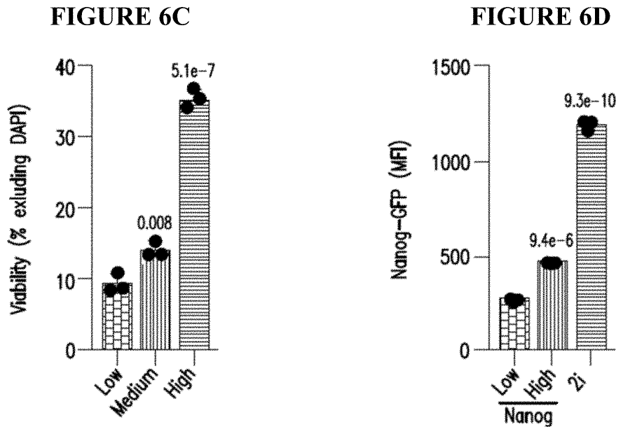

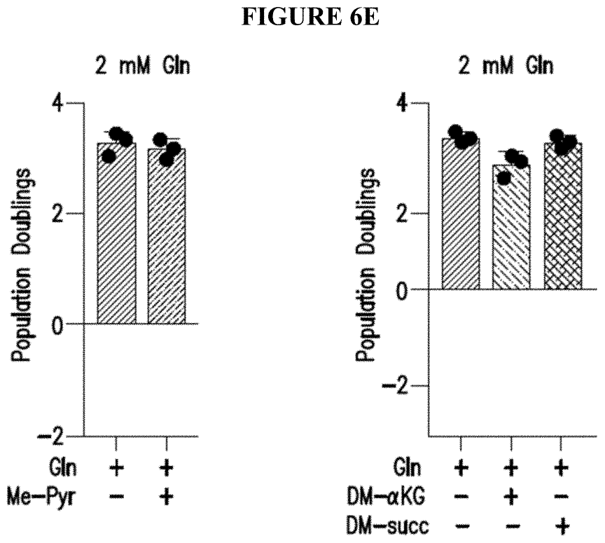

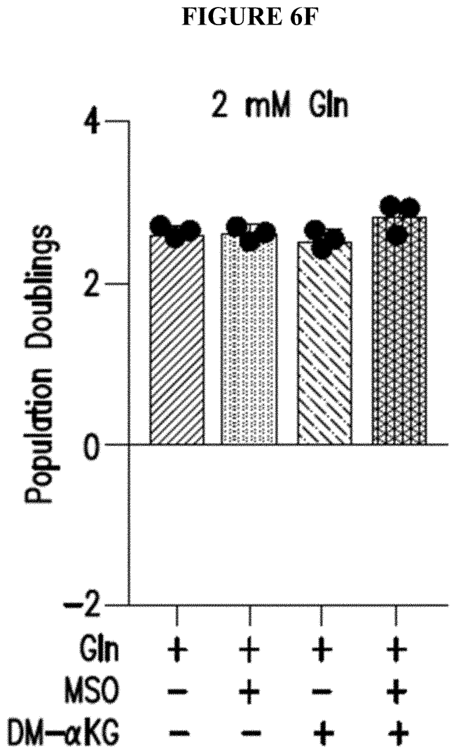

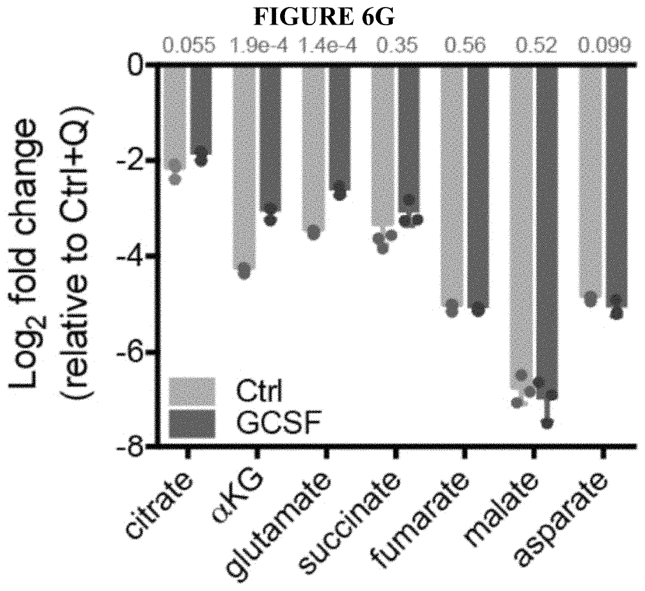

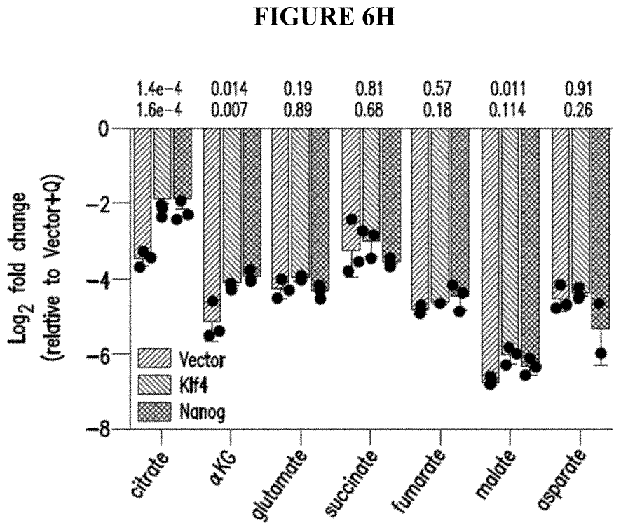

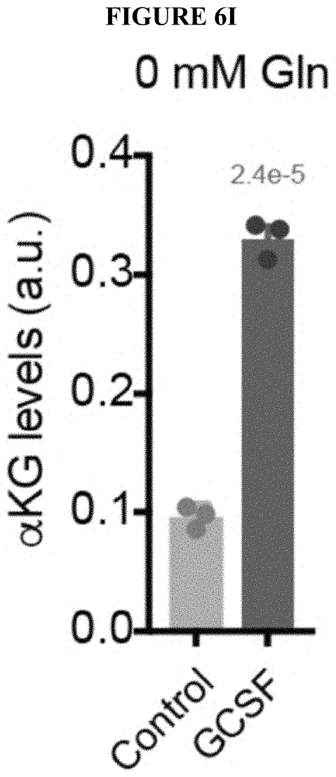

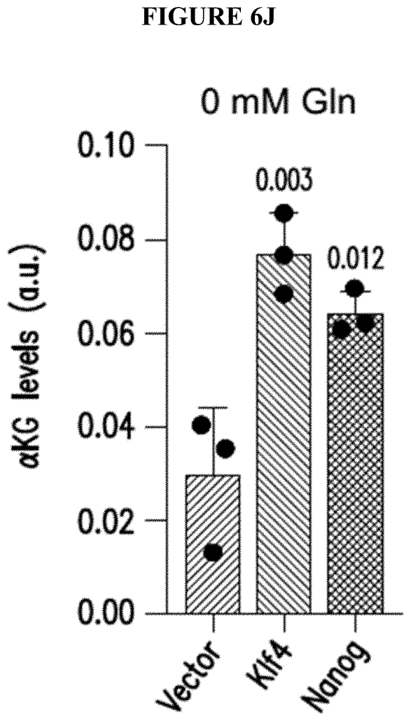

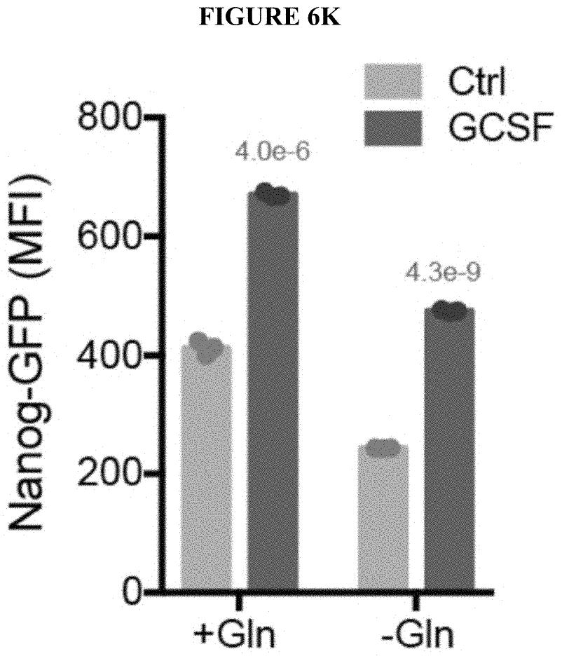

ESCs with enhanced self-renewal exhibit reduced dependence on exogenous glutamine. The present disclosure next probed the functional outcome of reduced glutamine anaplerosis in order to further test whether altered proliferative metabolism is an inherent feature of cells with enhanced capacity for self-renewal. Glutamine is required to maintain proliferation, viability and self-renewal of ESCs in traditional S/L culture, and restoring anaplerosis with cell-permeable αKG is sufficient to compensate for glutamine withdrawal (FIG. 2A and FIGS. 6A-6B). Therefore, the present disclosure reasoned that decreased reliance on glutamine anaplerosis would enable cells to better tolerate withdrawal of exogenous glutamine. Indeed, Nanog-high, Nanog-intermediate, and Nanog-low cells were progressively more sensitive to glutamine withdrawal (FIG. 6C). Even after several days in culture, “Nanog High” cells were significantly more resistant to apoptosis triggered by glutamine deprivation than their “Nanog Low” counterparts (FIG. 2B and FIG. 6D). As a control, the present disclosure included cells cultured in the presence of 2i, which is sufficient to enable glutamine-independent proliferation⁸ and blocked apoptosis induced by glutamine withdrawal (FIG. 2B and FIG. 6D). Similarly, while only 40% of control ESCs remained viable after 48 h of glutamine withdrawal, over 60% remained viable in ESCs with constitutive JAK/STAT3 activation (FIG. 2C). Furthermore, cells with enhanced self-renewal mediated by either JAK/STAT3 activation or Klf4/Nanog overexpression were able to increase the number of viable cells in the culture despite the absence of exogenous glutamine (FIGS. 2D and 2E). Conversely, pharmacologic inhibition of JAK/STAT3 signaling sensitized ESCs to glutamine deprivation (FIG. 2F).

In order to determine which metabolic substrates are limiting for survival under conditions of glutamine deprivation, the present disclosure cultured cells with cell-permeable analogs of pyruvate, αKG and succinate. Only αKG, the direct substrate for de novo glutamine biosynthesis, was capable of rescuing survival and proliferation in the absence of glutamine, and this rescue was contingent upon the ability of cells to use αKG to engage in de novo glutamine biosynthesis (FIGS. 2G and 2H, FIGS. 6E and 6F). Therefore, the present disclosure asked whether cells with enhanced self-renewal are better able to maintain αKG pools during conditions of glutamine withdrawal. In complete medium, even in cells with enhanced self-renewal, glutamine provides the dominant source of carbon for TCA cycle anaplerosis (FIGS. 1D and 1F). Glutamine withdrawal profoundly reduced steady-state levels of TCA cycle metabolites in all ESC lines tested (FIGS. 6G and 6H). However, both JAK/STAT3-activated and Klf4/Nanog-overexpressing cells were better than their control counterparts at sustaining intracellular pools of αKG, but not downstream TCA cycle metabolites (FIGS. 6G-6J). As a result, JAK/STAT3 and Klf4/Nanog-overexpressing cells were better able to maintain an elevated αKG/succinate ratio relative to control ESCs in the absence of exogenous glutamine (FIGS. 2I-2J).

In addition to serving as an obligate substrate for de novo glutamine biosynthesis, intracellular αKG promotes Nanog expression and increases self-renewal (Carey et al., Nature 518, 413-416 (2015); Hwang et al., Cell metabolism 24, 494-501 (2016); TeSlaa et al., Cell metabolism 24, 485-493 (2016)). Accordingly, while control cells rapidly lost Nanog expression upon glutamine deprivation such that both the overall Nanog expression and the proportion of cells in the Nanog-high population decreased, cells with JAK/STAT3 activation were able to retain Nanog expression and the fraction of cells in the Nanog-high population despite absence of exogenous glutamine (FIG. 2K, FIG. 6K). Together, these results indicate that stem cells with strengthened pluripotency networks are not only better able to survive glutamine deprivation but also able to withstand the destabilizing effect of loss of glutamine on markers of self-renewal.

The relative glutamine independence of cells with heightened self-renewal led us to speculate that the present disclosure could exploit this metabolic property to select for cells with the highest potential for self-renewal. To test this idea, the present disclosure performed competition assays in which GFP-tracked cells expressing empty vector, Klf4 or Nanog were mixed with parental ESCs and the proportion of GFP+ cells was assessed following 48 h culture in glutamine-replete or glutamine-free medium. Expression of Nanog or Klf4 resulted in a notable selective advantage in the absence of exogenous glutamine, such that the proportion of GFP+ cells increased by 41% (Nanog) or 69% (Klf4) relative to cells cultured in the continuous presence of glutamine (FIG. 2L).

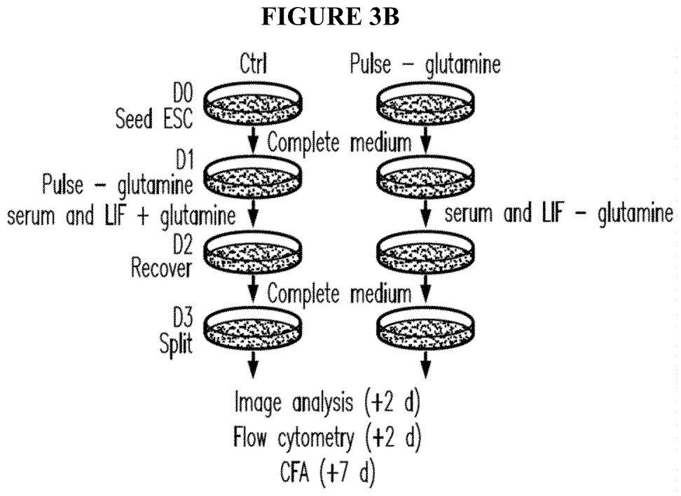

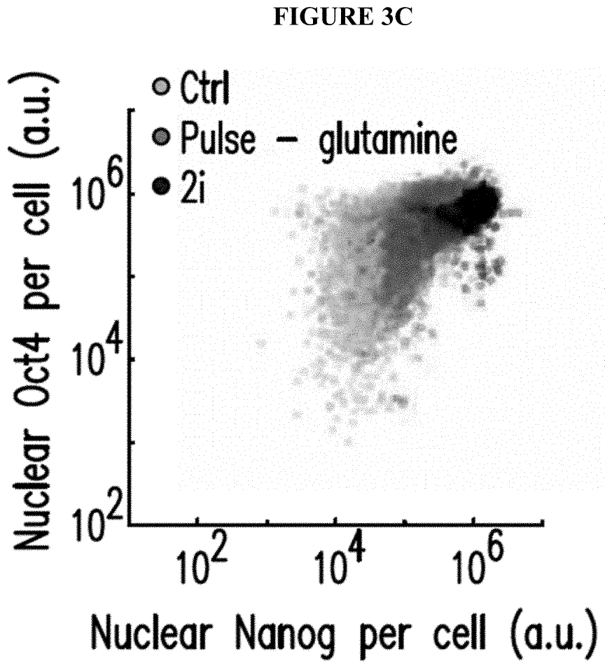

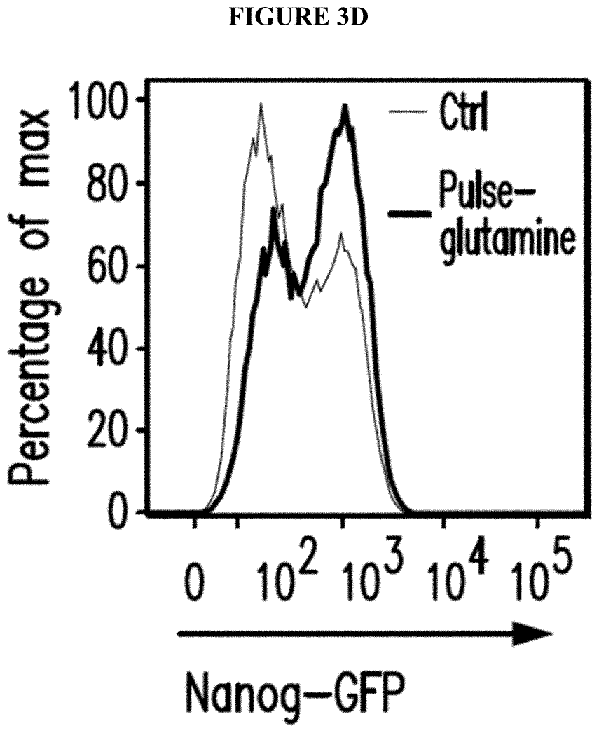

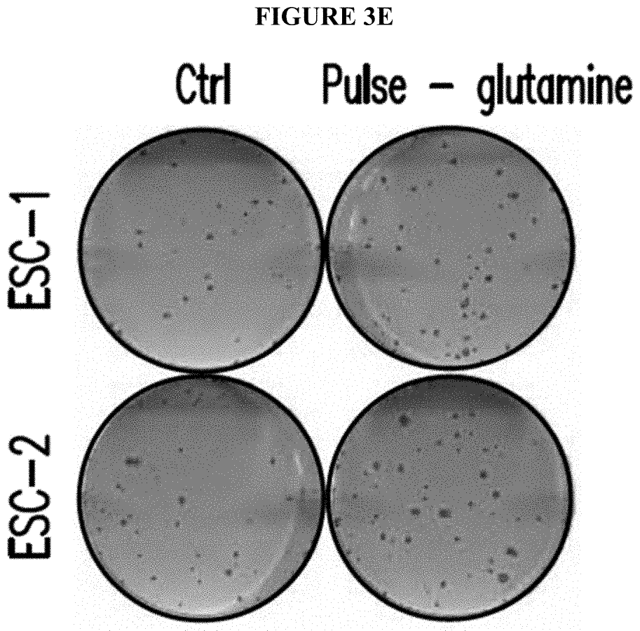

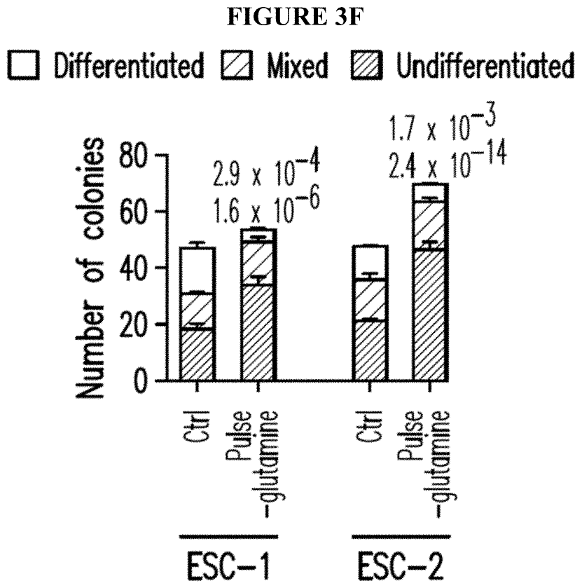

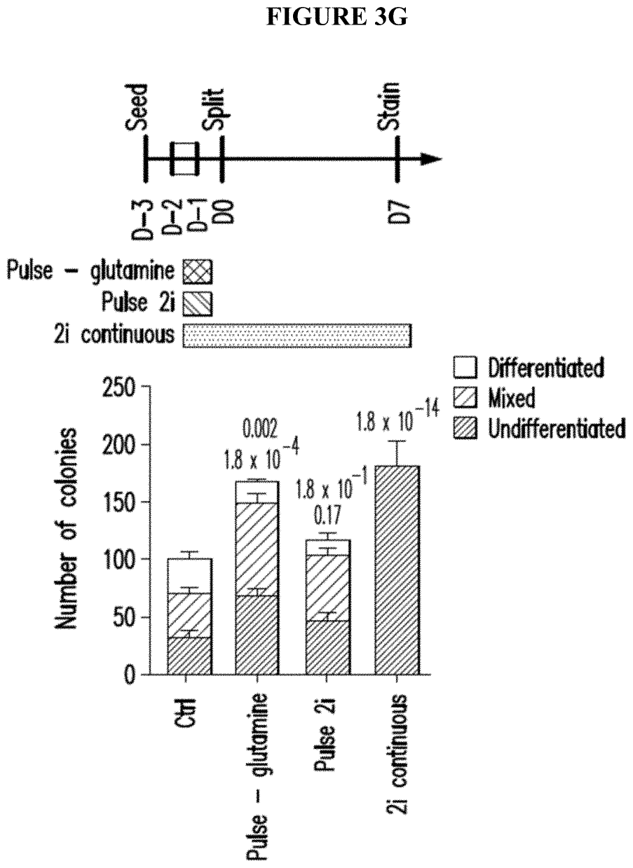

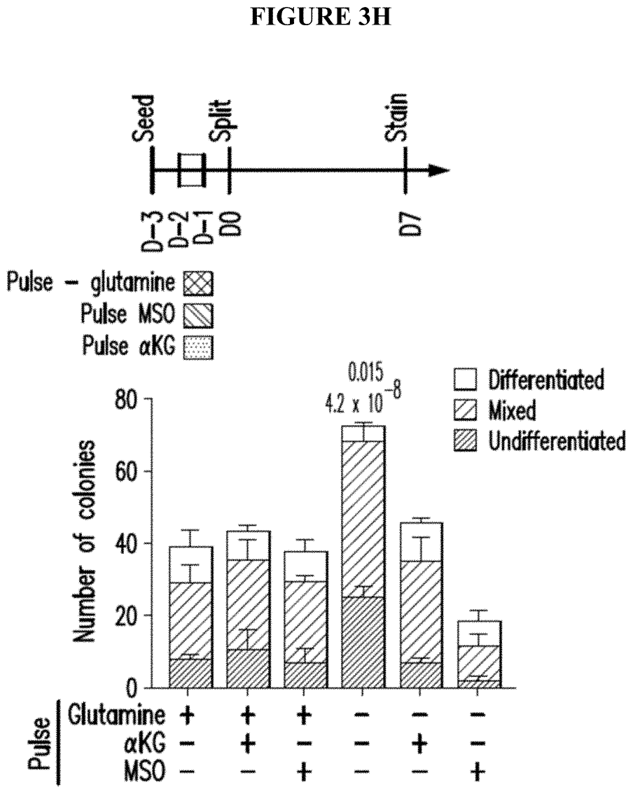

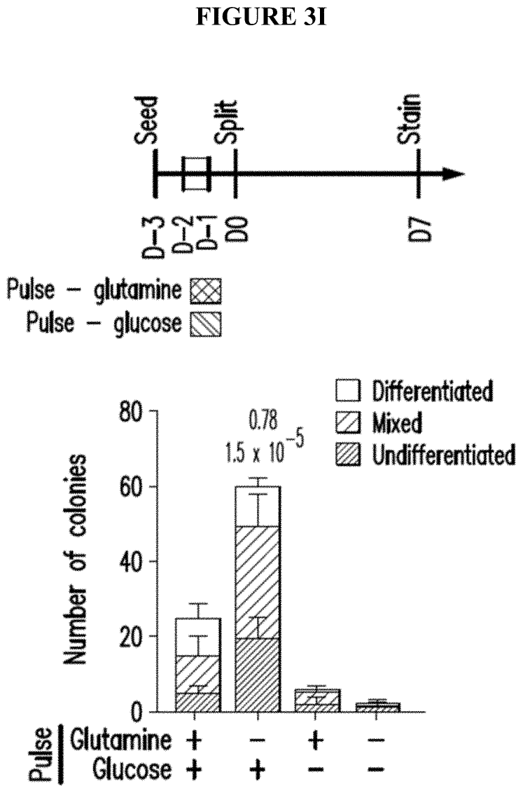

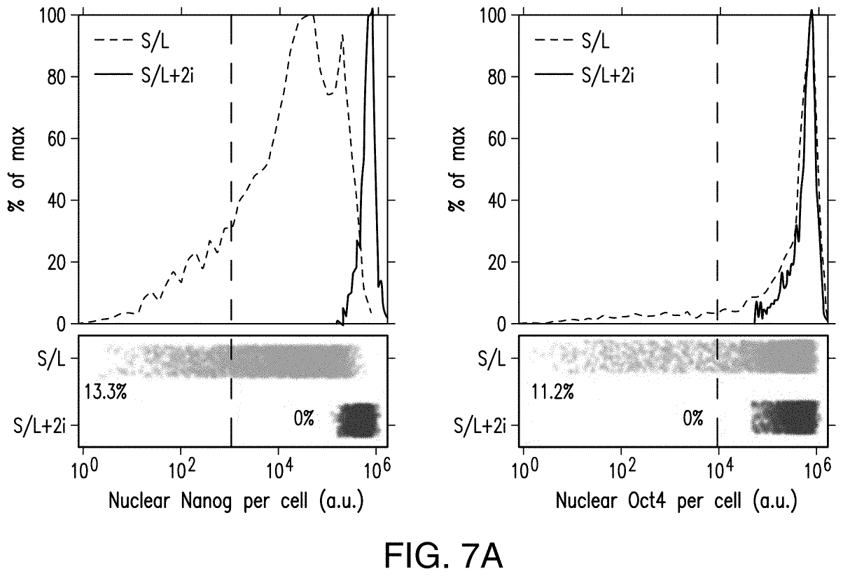

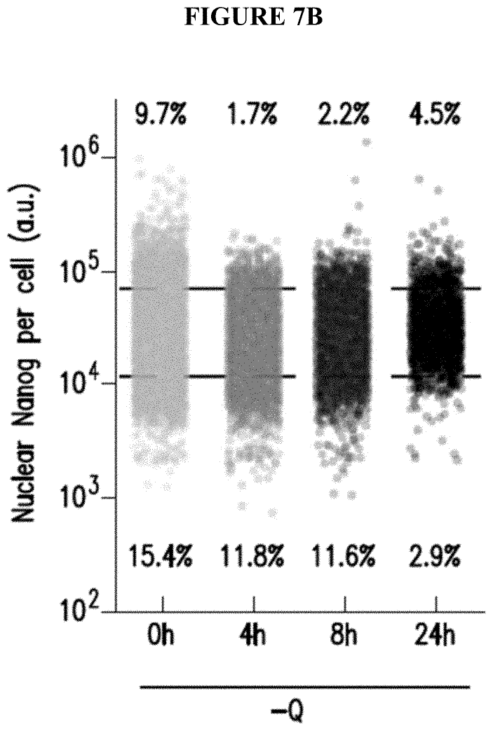

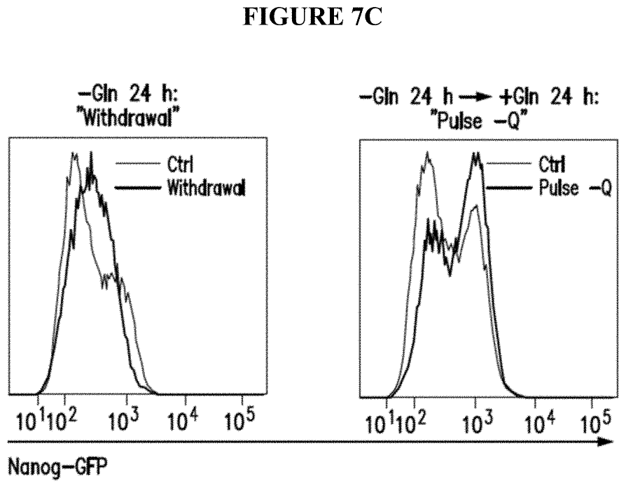

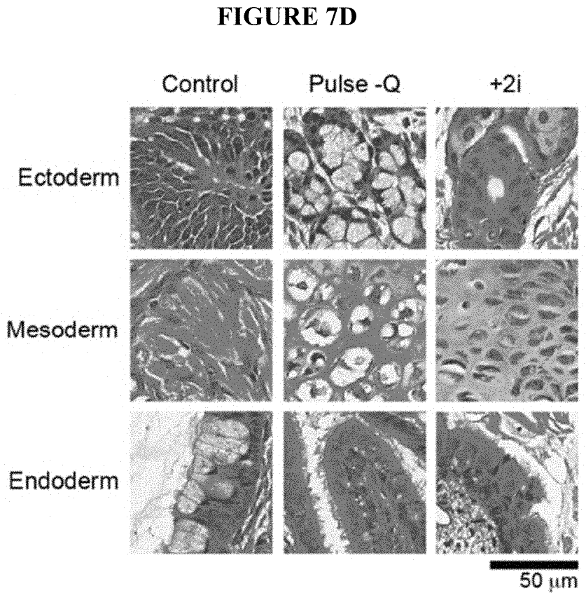

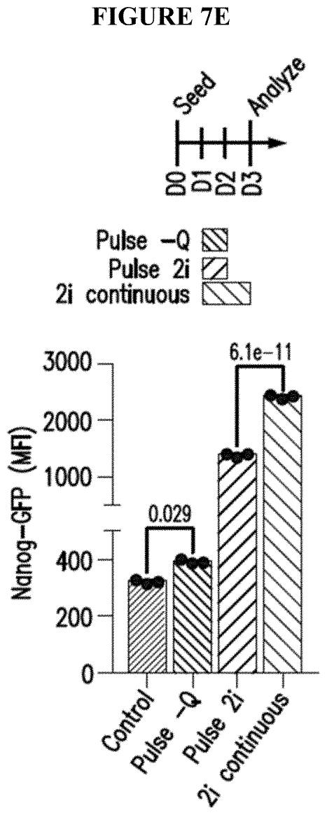

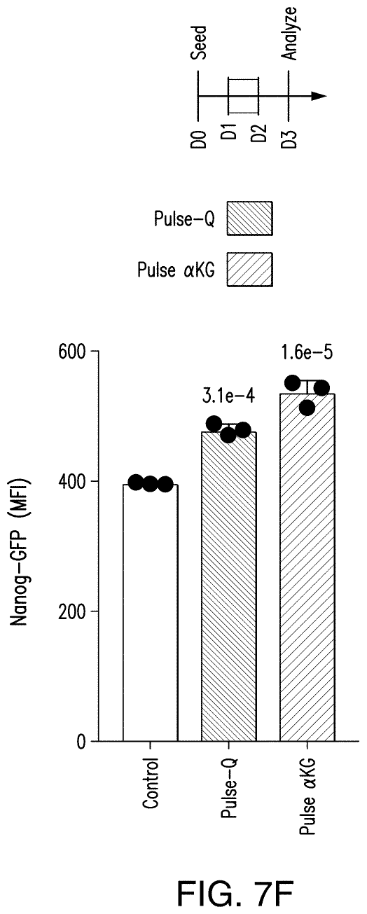

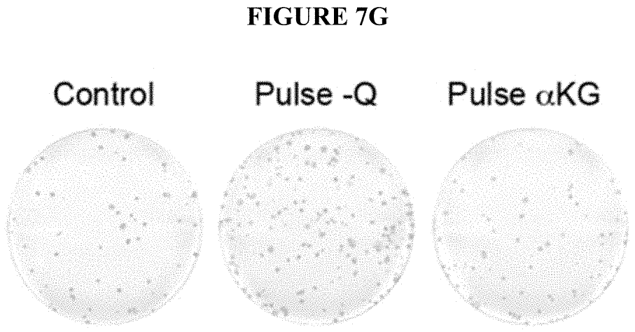

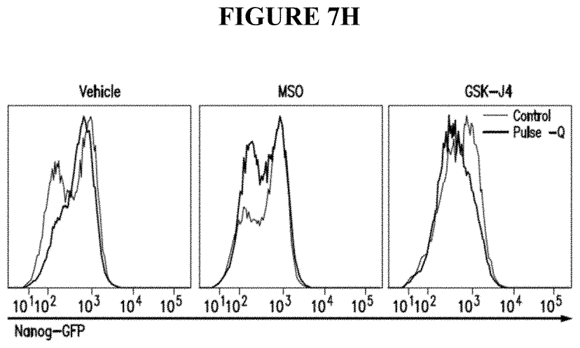

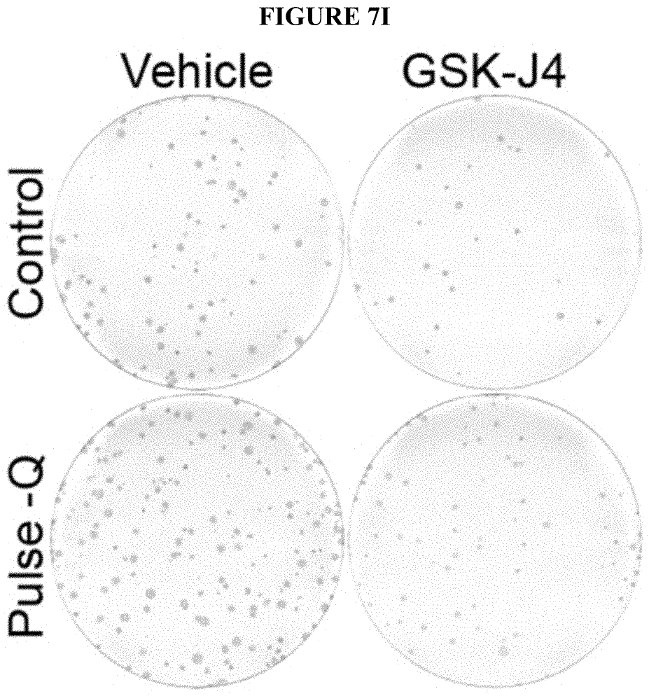

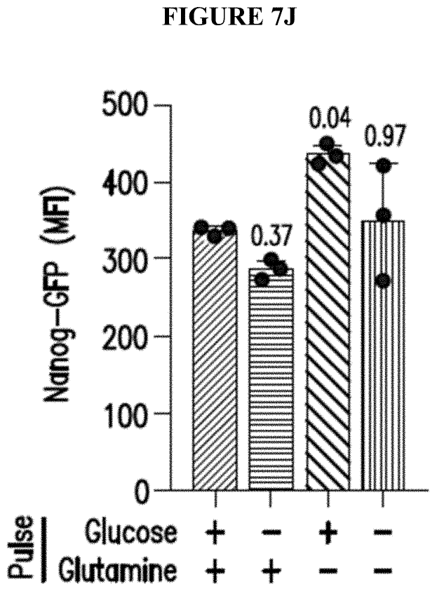

The present disclosure next asked whether glutamine depletion could select for cells with endogenously strengthened self-renewal potential from within the heterogeneous population characteristic of ESCs. In order to assess population heterogeneity, the present disclosure developed a quantitative immunofluorescence (IF)-based assay that allowed us to measure the expression levels of pluripotency-associated transcription factors in individual cells. Consistent with previous reports, IF analyses demonstrated that S/L-cultured ESCs exhibit highly variable Nanog expression and relatively homogenous, unimodal Oct4 expression (FIG. 7A) (Chambers et al., Cell 113, 643-655 (2003); Karwacki-Neisius et al., Cell Stem Cell 12, 531-545 (2013); Silva et al., Cell 138, 722-737 (2009)). Addition of 2i increased both homogeneity and overall level of Nanog expression while having minimal effects on mean Oct4 expression (FIG. 7A). Of note, 2i eliminated a long tail of “Oct4-low” cells representing approximately 10% of S/L-cultured ESC that have previously been reported to represent differentiated cells readily apparent in traditional S/L ESC cultures (Karwacki-Neisius et al., Cell Stem Cell 12, 531-545 (2013)) (FIG. 7A). As Oct4 is required for pluripotency (Nochols et al., Cell 95, 379-391 (1998)), Oct4-low cells represent the most committed cells within a heterogenous population. Therefore, the present disclosure asked whether these cells were the most susceptible to glutamine withdrawal. Indeed, within 8 hours of glutamine withdrawal, the proportion of cells in the Oct4-low fraction declined by 50% and 24 h of glutamine deprivation almost entirely eliminated the population of Oct4-low cells (FIG. 3A). Similarly, 24 h of glutamine deprivation eliminated the majority of Nanog-low cells; however, consistent with the observation that Nanog expression is sensitive to glutamine availability (FIG. 2K), glutamine deprivation also reduced the fraction of cells exhibiting the highest Nanog expression (FIGS. 7B and 7C).

While Nanog expression is metastable and Nanog-low cells can remain undifferentiated and regenerate Nanog-high cells, cells with very low Oct4 represent differentiated cells that cannot self-renew (Karwacki-Neisius et al., Cell Stem Cell 12, 531-545 (2013)). As these Oct4-low cells were sensitive to glutamine deprivation, the present disclosure hypothesized that transient glutamine deprivation would eliminate the most committed cells and thereby improve the overall self-renewal potential of a population. This simple procedure entailed subjecting regularly cultured ESCs to glutamine free medium for 24 h (“pulse”) and then recovering the cells in complete medium before seeding for follow-up experiments (FIG. 3B). Immunofluorescence assays confirmed that ESCs subjected to a 24-hour period of glutamine withdrawal had higher Nanog and Oct4 on a per-cell basis, reflecting elimination of the most committed cells and the restoration of Nanog levels following glutamine re-addition (FIG. 3C). Similarly, Nanog-GFP reporter cells demonstrated that “pulsed” ESCs were more likely to fall into the Nanog-high population than their conventionally cultured control counterparts (FIGS. 3D and 7C). In line with their enhanced expression of pluripotency-associated transcription factors, pulsed cells also exhibited enhanced self-renewal. Colony formation assays analyzed more than one week after the initial pulse demonstrated that pulsed cells were more likely to give rise to undifferentiated colonies and less likely to give rise to differentiated colonies (FIGS. 3E-3F). Importantly, ESCs subjected to transient glutamine withdrawal remained competent for multi-linage differentiation, giving rise to all three germ layers in vivo during teratoma formation (FIG. 7D).

The present disclosure next performed a series of experiments to clarify how transient glutamine withdrawal improves the self-renewal potential of a population of ESCs. The present disclosure first compared pulsed glutamine withdrawal, which eliminates the most committed cells, with interventions that increase ESC self-renewal. In contrast to pulsed glutamine deprivation, pulsed treatment with 2i or αKG—interventions that transiently increase Nanog-GFP expression (FIGS. 7E-7F)—had no durable effect on the self-renewal capacity of a population of ESC cells (FIGS. 3G and 7G). To confirm that selective elimination of cells that cannot survive glutamine deprivation is required for the benefit of the pulsed glutamine withdrawal, the present disclosure supplemented cells with cell-permeable αKG at the time of glutamine withdrawal. Blocking cell death during glutamine deprivation (FIG. 2G) eliminated the selective advantage of glutamine deprivation and abrogated the benefit of the pulse (FIG. 3H). Further supporting the notion that the glutamine withdrawal pulse selects for cells that have the endogenous metabolic capacity to sustain de novo glutamine synthesis, inhibition of glutamine synthetase concurrent with glutamine withdrawal eliminated surviving cells and completely blocked the ability of transient glutamine withdrawal to improve the population self-renewal capacity (FIGS. 3H and 7H).

Enhanced self-renewal is associated with the ability to sustain intracellular αKG in the absence of exogenous glutamine (FIGS. 61 and 6J). In addition to enabling glutamine synthesis, intracellular αKG can also promote the activity of αKG-dependent chromatin modifying enzymes. The ability of surviving ESCs to preserve intracellular αKG pools to maintain αKG-dependent demethylation reactions may also contribute to the beneficial effect of transient glutamine withdrawal, as addition of the histone H3 trimethylated lysine 27 (H3K27me3) demethylase inhibitor GSK-J4 impaired ESC self-renewal both when administered during transient glutamine withdrawal and in the presence of exogenous glutamine (FIGS. 7H and 7I). In contrast to glutamine, glucose is required for the viability of ESCs regardless of self-renewal capacity (Carey et al., Nature 518, 413-416 (2015)). Accordingly, pulsed glucose withdrawal strongly reduced the colony-formation capacity of mouse ESCs (FIGS. 3I and 7J). Moreover, in line with previous reports that combined glucose and glutamine withdrawal eliminates undifferentiated stem cells (Tohyama et al., Cell metabolism 23, 663-674 (2016)), pulsed withdrawal of both glucose and glutamine decimated the ability of ESCS to form colonies (FIG. 3I). Together, these findings support a model wherein glutamine deprivation selectively eliminates the most committed cells within a population thereby durably enhancing the overall self-renewal capacity of the remaining cells.

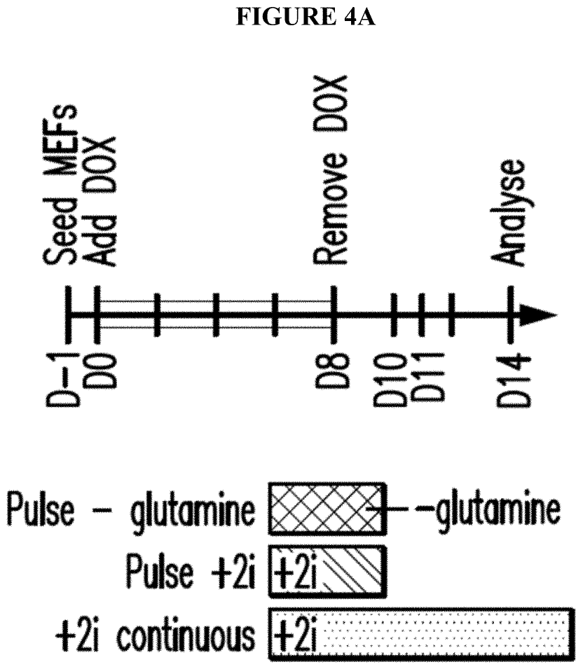

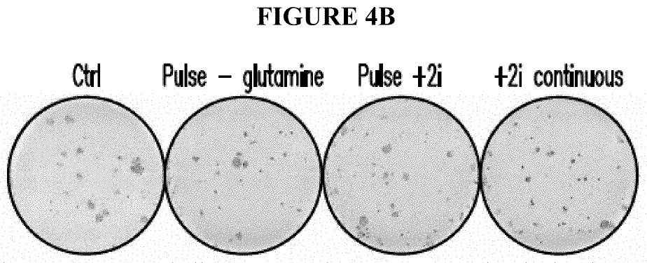

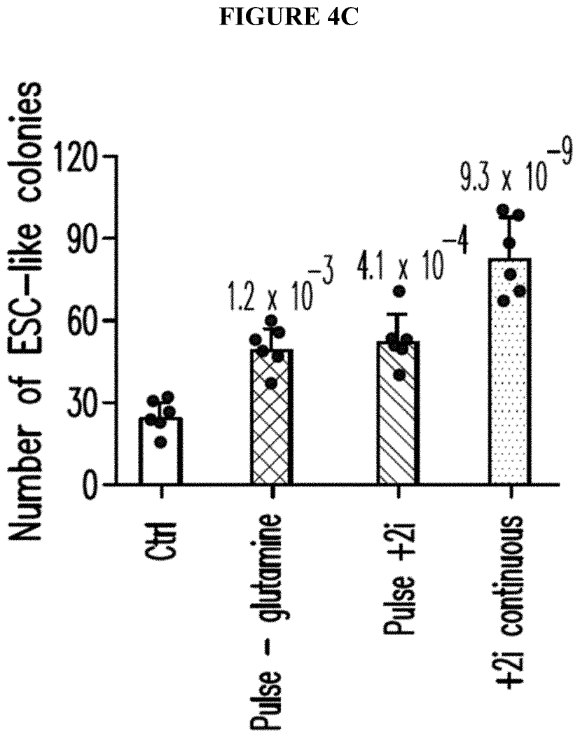

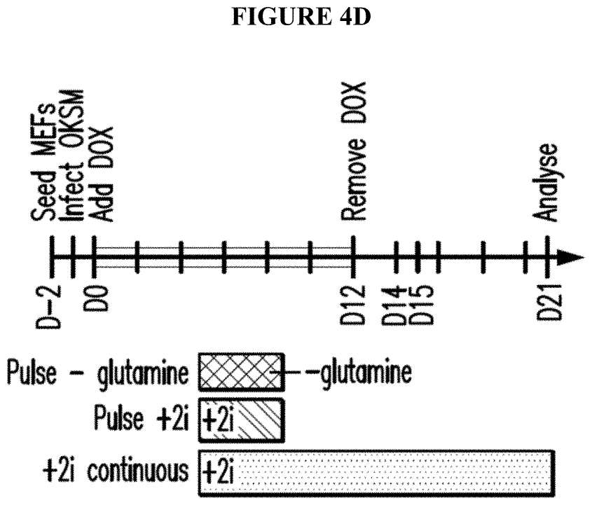

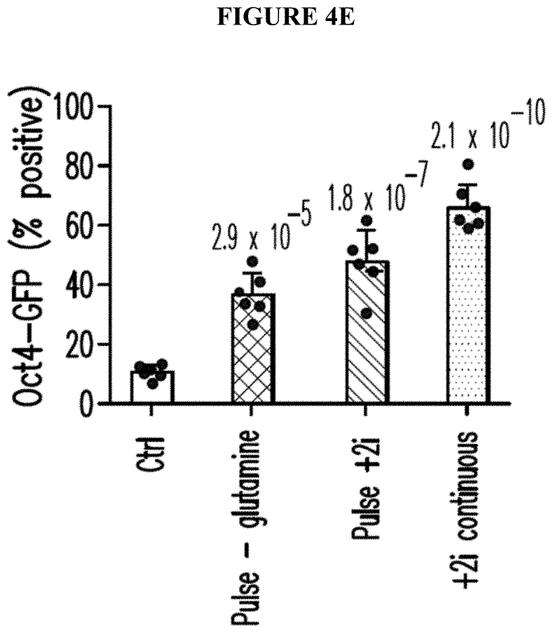

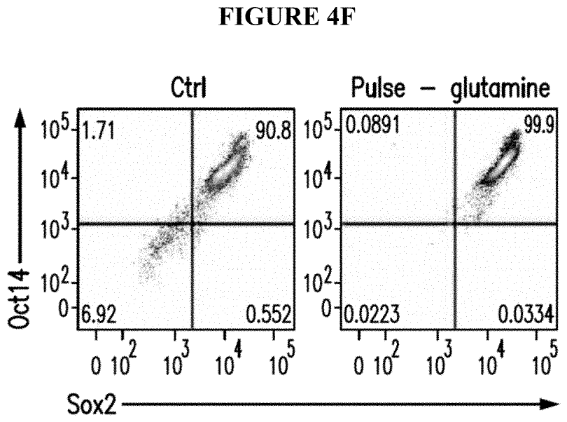

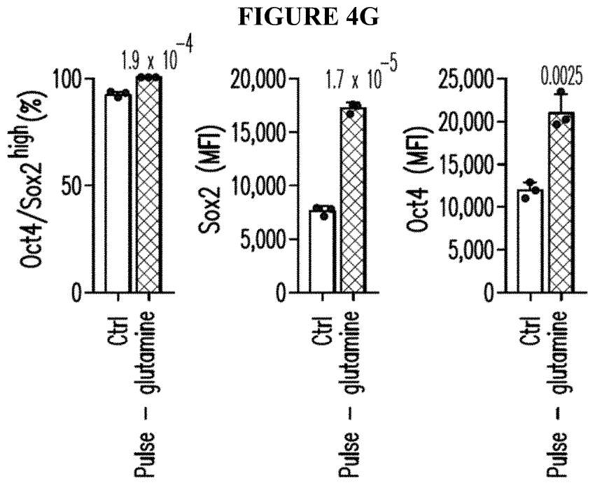

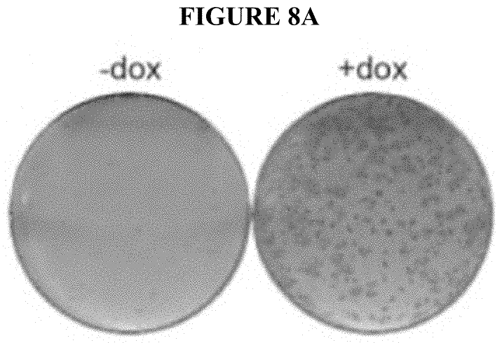

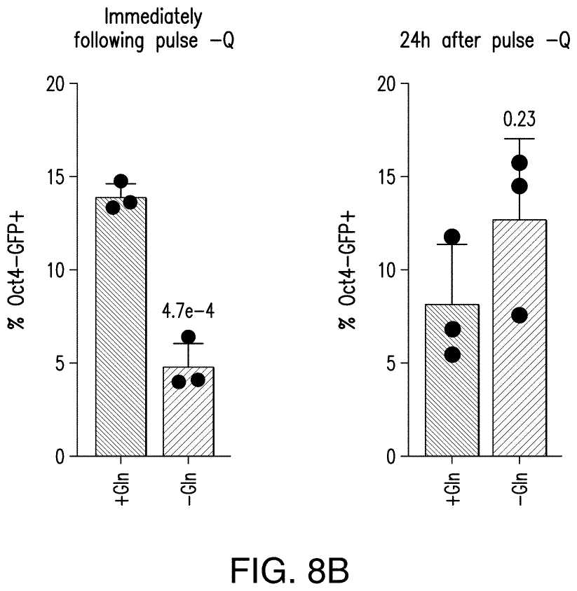

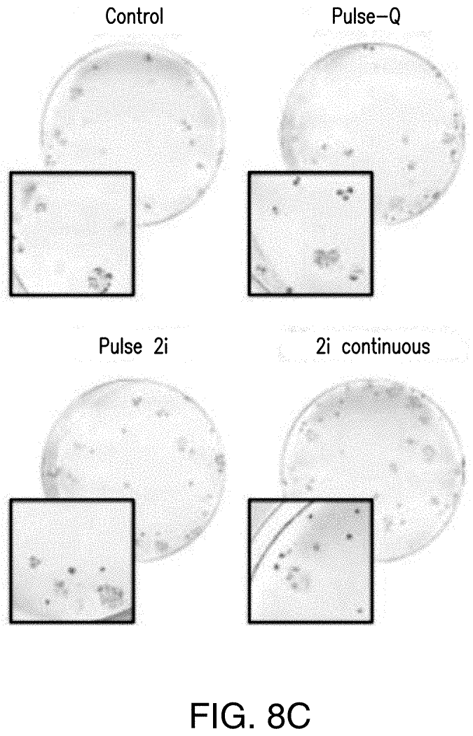

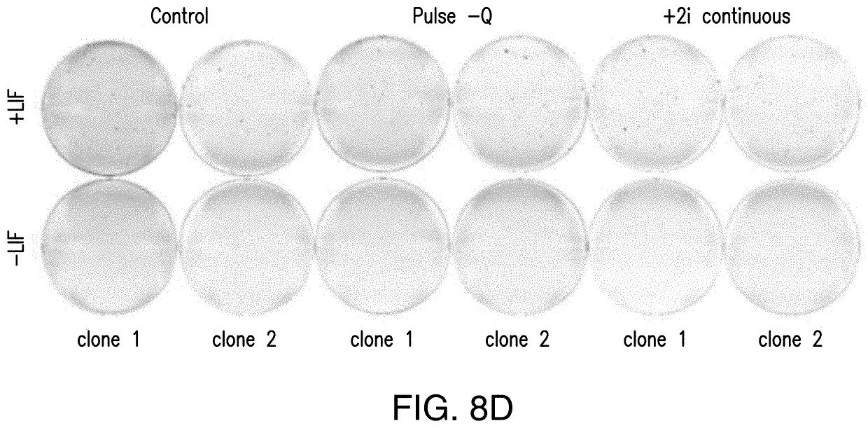

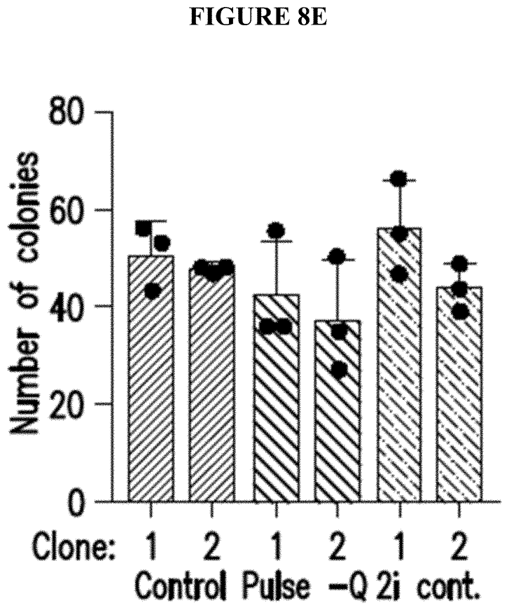

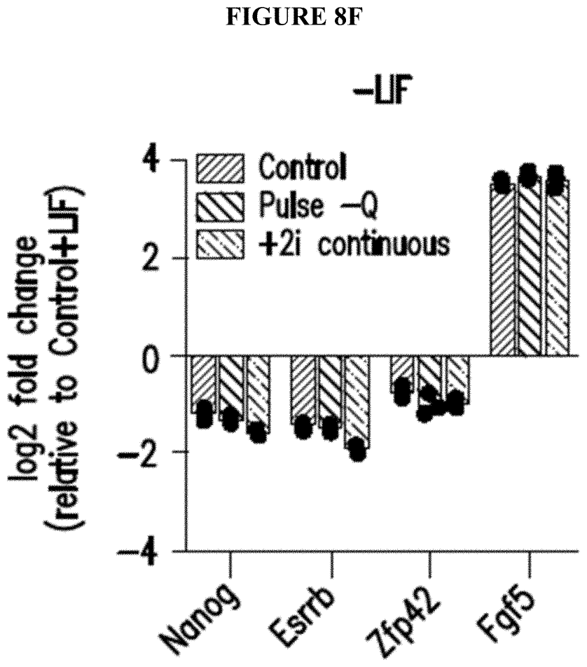

Transient glutamine withdrawal enhances mouse somatic cell reprogramming to pluripotency. Reprogramming of somatic cells to pluripotency represents a major area in which stem cell heterogeneity poses a significant experimental hurdle. Reprogramming is an inefficient process hampered by low efficacy and the persistence of incompletely reprogrammed cells (Hochedlinger et al., Cold Spring Harb Perspect Biol 7 (2015)). Interventions that consolidate the pluripotency network enhance reprogramming efficiency: for example, adding 2i to partially reprogrammed cells efficiently promotes the formation of fully reprogrammed cells (Silvia et al., PLoS Biol 6, e253 (2008)). Therefore, the present disclosure tested whether glutamine withdrawal, which selects for cells with strengthened pluripotency gene networks, improves reprogramming efficiency. First, the present disclosure utilized mouse embryonic fibroblasts (MEFs) harboring a polycistronic cassette enabling doxycycline (dox)-inducible expression of Oct4, Klf4, Sox2 and c-Myc (OKSM) (FIG. 4A) (Stadtfeld et al., Nat Methods 7, 53-55 (2010); Dun et al., Science 344, 1156-1160 (2014)). After eight days of dox-induced OKSM expression, dox removal forces cells to rely on reactivated endogenous pluripotency networks in order to sustain proliferation and ESC-like features including reactivity to alkaline phosphatase (AP). Consequently, cells that were never exposed to dox are fully AP-negative while control cells exposed to dox exhibit heterogeneous AP staining with numerous variably stained regions punctuated with discrete, well-stained colonies reminiscent of undifferentiated ESC colonies (FIG. 8A). As expected, sustained 2i treatment over the last four days of reprogramming, which helps cells induce and consolidate pluripotency transcriptional networks (Dun et al., Science 344, 1156-1160 (2014)), resulted in a marked increase in the number of discrete, ESC-like colonies (FIGS. 4B and 4C). Strikingly, just 24 h of glutamine deprivation two days after dox withdrawal was likewise sufficient to increase the overall number of ESC-like AP+ colonies (FIGS. 4B and 4C). Conversely, there was an overall reduction in flat, intermediately stained regions, consistent with selective elimination of cells with weak activation of pluripotency-associated gene networks (FIG. 4B). Notably, transient glutamine withdrawal was just as effective at enhancing reprogramming efficiency as transient treatment with 2i (FIGS. 4B and 4C).

To determine whether glutamine deprivation indeed increased the proportion of cells with activated endogenous pluripotency gene networks, the present disclosure utilized a second reprogramming system. Here, the present disclosure infected MEFs harboring a GFP reporter knocked into the endogenous Oct4 locus³⁹ with viruses carrying dox-inducible OKSM. The Oct4-GFP reporter is helpful in distinguishing fully reprogrammed iPSCs from partially reprogrammed “pre-iPSCs” which, despite having ESC-like morphology, do not activate endogenous pluripotency genes³⁶ and thus cannot ultimately maintain stable Oct4-GFP expression. Once again, the present disclosure subjected cells to sustained 2i (8 days) or a 24 h pulse of either glutamine deprivation (“Pulse −Q”) or 2i treatment (“Pulse 2i”) beginning 2 days after dox withdrawal (FIG. 4D). Consistent with the observation that glutamine withdrawal eliminates the most committed, Oct4-low cells, the number of Oct4-GFP+ cells decreased transiently during glutamine withdrawal but rebounded within 24 h of recovery in glutamine-replete medium (FIG. 8B). By the end of the experiment, all interventions significantly increased the proportion of cells expressing Oct4-GFP (FIG. 4E) and increased generation of tight, strongly AP+ ESC-like colonies (FIG. 8C). Once again, 24 h of glutamine withdrawal was as effective as 24 h of 2i treatment in enhancing generation of Oct4+iPSCs (FIGS. 4E and 8C). Consistent with being fully reprogrammed iPSCs, cells isolated from colonies with compact, ESC-like morphology sustained Oct4-GFP expression for multiple passages in vitro and were able to generate colonies from single cells (FIGS. 8D and 8E) while remaining sensitive to LIF withdrawal (FIGS. 8D and 8F). These data suggest that transient glutamine deprivation is sufficient to increase the fraction of somatic cells that undergo successful reprogramming and further reveal that a metabolic intervention is as effective as well-described alterations in signal transduction pathways at improving reprogramming efficiency.

Transient glutamine withdrawal increases markers of pluripotency in human ESCs. Finally, the present disclosure asked whether glutamine withdrawal exerted similar effects in human pluripotent stem cells despite the fact that human ESCs are cultured with dramatically different growth factors and represent a more committed, post-implantation stage of development (Weinberger et al., Nature reviews. Molecular cell biology 17, 155-169 (2016)). As with mouse ESCs, pulsed glutamine withdrawal eliminated a sub-population of cells with low expression of Oct4 (FIG. 4F). Moreover, pulsed glutamine withdrawal resulted in overall enhanced expression of key pluripotency factors Sox2 and Oct4 (FIGS. 4F and 4G). Thus, transient glutamine deprivation represents a general method to enhance expression of key pluripotency markers in both mouse and human pluripotent stem cells under a variety of culture conditions.

### DISCUSSION

The present disclosure establishes a distinct metabolic phenotype of naïve mouse embryonic stem cells—reduced reliance on extracellular glutamine as an anaplerotic substrate—is a generalizable feature of cells with enhanced self-renewal. Enhancing ESC self-renewal, either through manipulation of signal transduction or pluripotency-associated transcription factors, is sufficient to alter cellular metabolism to support enhanced survival in the absence of exogenous glutamine. Conversely, cells with weak pluripotency-associated transcription networks are highly glutamine dependent and rapidly die in the absence of exogenous glutamine supplementation. This association between glutamine dependence and pluripotency offers a potent, non-invasive and reversible method to select for stem cells from a heterogeneous population without altering the biological properties of any individual cell. Recent reports demonstrating potential negative effects of other established methods to enhance ground state pluripotency underscore the potential value of this strategy (Choi et al., Nature 548, 219-223 (2017); Yagi et al., Nature 548, 224-227 (2017)). Moreover, the generalizability of this method to human ESCs, in which the conditions required to achieve the naïve ground state remain a source of continued investigation (Weinberger et al., Molecular cell biology 17, 155-169 (2016)) underscores the potential utility of exploiting common metabolic features of cells with enhanced self-renewal.

The molecular drivers of reduced glutamine dependence in pluripotent stem cells remain to be fully elucidated. The subtly different effects of the various interventions that increase self-renewal on ESC metabolism may arise as a result of specific transcriptional profiles driven by each intervention or from additional consequences of altered signalling, such as mitochondrial translocation of STAT3 (Carbognin, E. et al., EMBO J. 35, 618-634 (2016)). It was previously demonstrated that reduced glutamine anaplerosis enables naïve ESCs to maintain high levels of αKG, a critical co-substrate for demethylation reactions that are required to maintain the unique chromatin landscape of naïve ESCs Carey et al., Nature 518, 413-416 (2015); Hwang et al., Cell metabolism 24, 494-501 (2016); TeSlaa et al., Cell metabolism 24, 485-493 (2016)). This consequence of reduced glutamine oxidation may provide a general advantage for mouse pluripotent stem cells, particularly given that pluripotency transcription factor binding of DNA is highly associated with local DNA demethylation during the establishment of ground state pluripotency (Ficz et al., Cell stem cell 13, 351-359 (2013); Habibi et al., Cell stem cell 13, 360-369 (2013)) and that fluctuations in glutamine-derived αKG levels have profound implications for maintenance of pluripotency (Hwang et al., Cell metabolism 24, 494-501 (2016); TeSlaa et al., Cell metabolism 24, 485-493 (2016)). However, decreased glutamine anaplerosis may provide additional advantages to naïve ESCs, independent of αKG. The ability to oxidize either glucose or glutamine to maintain energy homeostasis may be of particular value under conditions when either glucose or glutamine becomes limiting (Bauer et al., FASEB J 18, 1303-1305, doi:10.1096/fj.03-1001fje (2004); Frauwirth et al., Immunity 16, 769-777 (2002)). Furthermore, decreased glutamine anaplerosis may facilitate the utilization of glutamine for other purposes, including glutamate-dependent uptake of non-essential amino acids (Utsunomiya-Tate et al., J Biol Chem 271, 14883-14890 (1996)) as well as nucleotide biosynthesis (Kammen, et al., Biochim Biophys Acta 30, 195-196 (1958)). Finally, glutamine not used as an anaplerotic substrate can be utilized for the synthesis of glutathione, which is essential to prevent cysteine oxidation and degradation of Oct4 in human ESCs (Marsboom et al., Cell Rep 16, 323-332 (2016)).

Prior studies have identified selective nutrient dependencies that can be exploited to preferentially eliminate ESCs from a population (Alexander et al., Proc Natl Acad Sci USA 108, 15828-15833 (2011)). The presently disclosed findings offer a strategy for the preferential enrichment of highly self-renewing, pluripotent ESCs. These results add to an increasing body of work supporting the idea that individual cell types may engage in distinct modes of nutrient use to support diverse cell fate decisions, including proliferation and regulation of gene expression. Further study of the specialized metabolism of individual cell types may deepen the understanding of how nutrient availability can affect cell fate decisions in vivo and provide further opportunities for intervention to select for cells with desired phenotypes.

### Methods

### Cell Culture

Mouse ESC lines (ESC1, ESC2) were generated from C57BL/6×129S4/SvJae F1 male embryos as previously described⁸. Nanog-GFP reporter ESCs were a gift from R. Jaenisch (MIT). Nanog-GFP lines expressing the chimeric LIF receptor and ESC1 lines overexpressing Nanog or Klf4 were generated as previously described (Finley et al., Nat Cell Biol 20, 565-574 (2018)). ESC1 cells were used for all experiments unless otherwise noted. ESCs were maintained on gelatin-coated plates in serum/LIF (S/L) medium containing Knockout DMEM (Life 10829-018) supplemented with 10% ESC-qualified FBS (Gemini), 0.1 mM 2-mercaptoethanol, 2 mM L-glutamine and 1000 U/mL LIF (Gemini). For culture in 2i (S/L+2i), S/L medium was supplemented with 3 μM CHIR99021 (Stemgent) and 1 μM PD0325901 (Stemgent). Cells were adapted to 2i or GCSF (Gemini) by passaging cells in S/L+2i or S/L+GCSF medium at most three times prior to use in experiments. S/L+2i-adapted cells were maintained for a maximum of nine passages. For human embryonic stem cells (hESC) culture, an H1 hESC line (NIHhESC-10-0043) with a previously described inducible Cas9 insertion was used (DeBerardinis et al., Cell metabolism 7, 11-20 (2008)). This line was maintained in chemically defined, serum-free E8 conditions (Thermo Fisher Scientific, A1517001) on tissue culture treated polystyrene plates coated with vitronectin (Thermo Fisher Scientific, A14700). hESCs were split with 0.5 mM EDTA at a 1:10-1:20 split ratio every 3-5 days. Cells have been confirmed to be mycoplasma-free by the MSKCC Antibody and Bioresource Core Facility. All experiments were approved by the Tri-SCI Embryonic Stem Cell Research Oversight Committee (ESCRO).

### Nutrient Deprivation Experiments

For glutamine deprivation experiments in mouse ESCs, cells were initially plated in standard S/L medium as described above. The following day, cells were washed with PBS and then cultured in experimental medium containing a 1:1 mix of glutamine-free DMEM (Gibco 11960-051) and glutamine-free Neurobasal medium (Gibco 21103-049) including 10% dialyzed FBS, 2-mercaptoethanol, and LIF as described above and containing (“+Q”) or lacking (“−Q”) L-glutamine (2 mM) as indicated. When indicated, dimethyl-α-ketoglutarate (Sigma 349631) dissolved in DMSO was added to a final concentration of 4 mM. For glucose and glutamine deprivation experiments, cells were cultured in medium containing a 1:1 mix of glutamine and glucose-free DMEM (Gibco A14430-01) and glutamine and glucose-free Neurobasal-A medium (Gibco A24775-01) including 10% dialyzed FBS and all supplements as described above, and containing or lacking glucose or glutamine as indicated.

### Embryonic Stem Cell Competition Assays.

GFP-negative parental ESCs were mixed with GFP-positive vector or Klf4/Nanog-overexpressing transgenic ESCs and seeded at a concentration of 30,000 total cells per well of a 12-well plate in triplicate. The following day, cells were washed with PBS and then changed to experimental medium containing a 1:1 mix of glutamine-free DMEM and glutamine-free Neurobasal medium including 10% dialyzed FBS, 2-mercaptoethanol, LIF, and containing (“+Q”) or lacking (“−Q”) L-glutamine as indicated. After 48 hours, cells were trypsinized for flow cytometry analysis. Cells were evaluated for GFP and DAPI on either a LSRFortessa or LSR-II machine (Beckman Dickinson). Analysis of DAPI exclusion and GFP mean fluorescence intensity was performed using FlowJo v9.0.

### Glutamine Pulse Experiments

For transient glutamine withdrawal (“pulse”) experiments, cells were initially plated in standard S/L medium as described above. The following day, cells were washed with PBS and then changed to experimental medium containing a 1:1 mix of glutamine-free DMEM and glutamine-free Neurobasal medium including 10% dialyzed FBS, 2-mercaptoethanol, LIF, and containing (“Ctrl”) or lacking (“Pulse”) L-glutamine as indicated. 24 hours later, cells were washed with PBS and then returned to glutamine-replete medium (“Recover”). 24 hours later, cells were subjected to either image analysis, flow cytometry, or plated for colony formation assays as indicated. For glutamine pulse experiments in human ESCs, cells were initially plated at a density of 300,000 cells/well in a tissue-culture treated polystyrene 12-well plate coated with vitronectin (Thermo Fisher Scientific, A14700) in E8 medium (Thermo Fisher Scientific, A1517001) containing 10 uM ROCK inhibitor Y-27632 (Selleck Chemicals S1049). 24 hrs after plating, medium was changed to modified E8 medium containing: DMEM high glucose without glutamine (Thermo Fisher 11960044), 10.7 mg/L Transferrin (Sigma T0665), 64 mg/L L-Ascorbic Acid (Sigma A890), 14 ug/L Sodium Selenite (Sigma S5261), 543 mg/L Sodium Bicarbonate (Research Products International 144558), 19.4 mg/L insulin (Sigma 19278), 100 ug/L bFGF (EMD Millipore GF003AF), 2 ug/L TGFβ1 (Peprotech 10021), and 2 mM L-glutamine. After 24 hrs of culture in modified E8 medium, medium was changed to modified E8 medium containing 2 mM or 0 mM L-glutamine for 24 hrs. All cells were then changed to modified E8 medium containing 2 mM L-glutamine and cultured for 24 hrs before harvest for analysis.

### Growth Curves

ESCs were seeded at a density of 30,000-40,000 cells per well of a 12-well plate. The following day, three wells of each line were counted to determine the starting cell number. The remaining cells were washed with PBS and cultured in medium containing a 1:1 mix of glutamine-free DMEM and glutamine-free Neurobasal medium including 10% dialyzed FBS, 2-mercaptoethanol, LIF, and containing or lacking L-glutamine or glucose as indicated and with or without the addition of additional supplements as indicated. Dimethyl-alpha ketoglutarate and dimethyl-succinate were added at 4 mM. Methyl pyruvate was added at 2 mM. Ruxolitinib was added at 500 nM. Methyl sulfoximine was added at 200 nM. Cells were counted on the indicated days thereafter using a Beckman Multisizer 4e with a cell volume gate of 400-10,000 fL. Cell counts were normalized to starting cell number. All curves were performed at most two independent times.

### Colony Formation Assays

Cells were seeded at 200 cells per well in six-well plates in standard S/L medium. Medium was refreshed every 2-3 days. Six to seven days after initial seeding, wells were fixed with citrate/acetone/3% formaldehyde for 30 seconds and stained using the Leukocyte Alkaline Phosphatase Kit (Sigma) according to manufacturer instructions. For ESC colony formation assays, colonies were scored manually in a blinded fashion. For reprogramming experiments, alkaline-phosphatase colonies were scored automatically using ImageJ (NIH). Briefly, images of each well were binarized using the Default function and particles greater than 8 pixels with a circularity of 0.7-1.0 were counted.

### Fluorescence Activated Cell Sorting

Evaluation of cell viability and Nanog-GFP expression. Nanog-GFP ESCs³⁰ were seeded at a concentration of 40,000 cells per well of a 12-well plate. The next day, cells were washed with PBS and medium was changed to experimental medium containing a 1:1 mix of glutamine-free DMEM and glutamine-free Neurobasal medium including 10% dialyzed FBS, 2-mercaptoethanol, LIF, and containing (“+Q”) or lacking (“−Q”) L-glutamine as indicated. On the day of analysis, cells were trypsinized and resuspended in FACS buffer (PBS+2% FBS+1 mM EDTA) containing DAPI (1 μg/mL). Cells were evaluated for GFP and DAPI on either a LSRFortessa or LSR-II machine and FACSDiva software (Beckman Dickinson). Viable cells were those excluding DAPI (100-fold less than DAPI-positive cells). Nanog-GFP expression was measured by GFP mean fluorescence intensity and quantified using FlowJo v9.0. All experiments were performed at most two independent times.

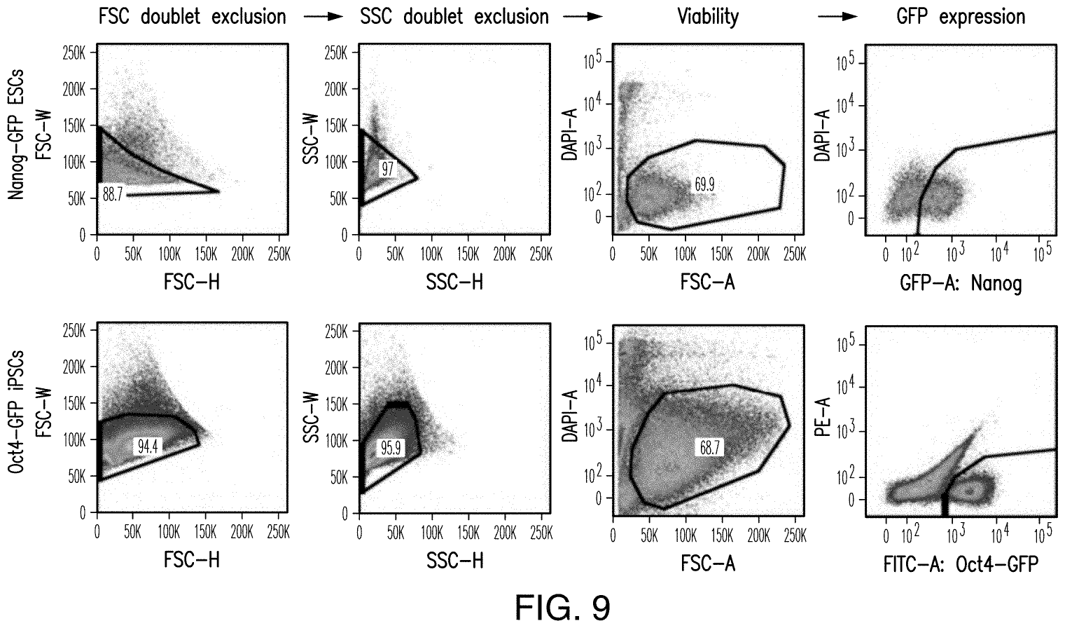

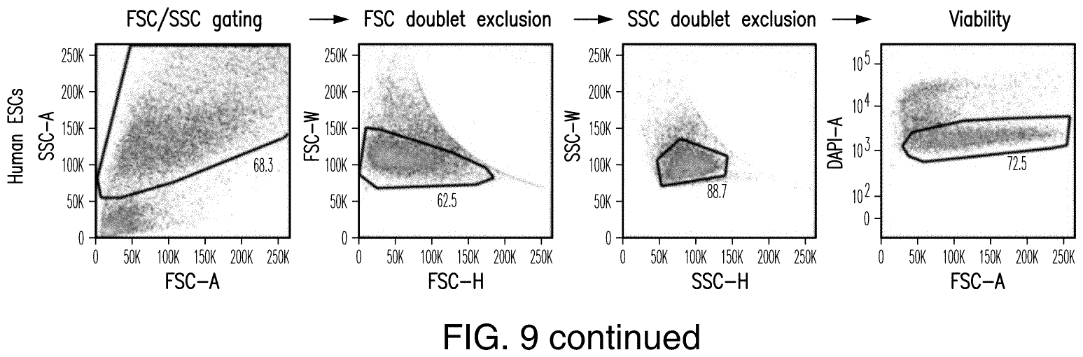

Sorting of Nanog High and Nanog Low populations. Gating strategy for fluorescence activated cell sorting analysis is shown in FIG. 9. Nanog-GFP ESCs that were cultured either in S/L medium or adapted to S/L+2i medium as described above were resuspended in sterile FACS buffer containing DAPI. DAPI-excluding cells were evaluated for Nanog-GFP expression on a BD FACSAria III cell sorter (Beckman Dickinson). “Nanog High” and “Nanog Low” populations were sorted based on Nanog-GFP expression levels in the highest 10% and lowest 10% of the population, respectively. Following sorting, cells were washed 2× with PBS to remove any residual FACS buffer and plated in 6-well gelatin-coated plates in standard S/L medium with the addition of penicillin/streptomycin (Life technologies). Cells were used 48 to 72 hours later for experiments.

Evaluation of apoptosis. Evaluation of apoptosis was performed using an Annexin V Apoptosis Detection kit (BD Biosciences BDB556570). Nanog-GFP ESCs that had been sorted based on Nanog-GFP expression 48 hours earlier as described above were plated in standard S/L medium. 24 hours later, cells were washed with PBS and cultured in experimental medium containing a 1:1 mix of glutamine-free DMEM and glutamine-free Neurobasal medium including 10% dialyzed FBS, 2-mercaptoethanol, LIF, and containing (“+Q”) or lacking (“−Q”) L-glutamine as indicated. 24 hours later, cells were trypsinized and resuspended in Annexin V binding buffer containing FITC-conjugated Annexin V and propidium iodide (PI) for 15 minutes at room temperature. Subsequently, excess binding buffer was added and both FITC and PI fluorescence was assessed on an LSRFortessa machine (Benton Dickinson). Apoptosis was quantified as cells positive for Annexin V based on a 100-fold increase in fluorescence as compared to negative cells.

Evaluation of Oct4 and Sox2 expression in human ESCs. Cells were dissociated using TryPLE-Select (Thermo Fisher 12563029) and resuspended in FACS buffer (5% FBS and 5 mM EDTA in FBS). Cells were first stained with LIVE/DEAD Violet (Molecular Probe, L34955, 1:1,000) for 30 minutes at RT. Cells were fixed and permeabilized in 1× fix/perm buffer (eBioscience, 00-5523-00) for 1 hr at RT. Cells were then stained with fluorophore conjugated antibodies OCT4-APC (eBioscience 50-5841-82, 1:25) and SOX2-Alexa488 (eBioscience 53-9811-82, 1:100) in permeabilization buffer (Thermo Fisher 00-8333-56) for 30 minutes at RT. Cells were washed by addition of FACS buffer and centrifugation between all steps. Analysis was performed after resuspension in FACs buffer using a BD Fortessa.

### Metabolic Analyses

Steady state TCA cycle metabolite measurements. Cells were seeded in standard S/L medium in 6-well plates. 24 hours later, cells were washed with PBS and changed into experimental medium containing a 1:1 mix of glutamine-free DMEM and glutamine-free Neurobasal medium including 10% dialyzed FBS, 2-mercaptoethanol, LIF, and 2 mM L-glutamine. The next day, cells were washed with PBS and subjected to the same experimental medium either with or without 2 mM glutamine. 8 hours later, metabolites were extracted with 1 mL ice-cold 80% methanol containing 2 μM deuterated 2-hydroxyglutarate (D-2-hydroxyglutaric-2,3,3,4,4-d₅ acid, d5-2HG) as an internal standard. After overnight incubation at −80° C., lysates were harvested and centrifuged at 21,000 g for 20 minutes to remove protein. Extracts were dried in an evaporator (Genevac EZ-2 Elite) and resuspended by incubating at 30° C. for 2 hours in 50 μL of 40 mg/mL methoxyamine hydrochloride in pyridine. Metabolites were further derivatized by addition of 80 μL of MSTFA+1% TCMS (Thermo Scientific) and 70 μl ethyl acetate (Sigma) and then incubated at 37° C. for 30 minutes. Samples were analyzed using an Agilent 7890A GC coupled to Agilent 5977C mass selective detector. The GC was operated in splitless mode with constant helium gas flow at 1 mL/min. 1 μl of derivatized metabolites was injected onto an HP-5MS column and the GC oven temperature ramped from 60° C. to 290° C. over 25 minutes. Peaks representing compounds of interest were extracted and integrated using MassHunter software (Agilent Technologies) and then normalized to both the internal standard (d5-2HG) peak area and protein content of duplicate samples as determined by BCA protein assay (Thermo Scientific). Ions used for quantification of metabolite levels are as follows: d5-2HG m/z 354; αKG, m/z 304; aspartate, m/z 334; citrate, m/z 465; fumarate, m/z 245; glutamate, m/z 363; malate, m/z 335 and succinate, m/z 247. All peaks were manually inspected and verified relative to known spectra for each metabolite.

Isotope tracing studies. For isotope tracing studies, cells were seeded in standard S/L medium in 6-well plates. 24 hours later, cells were washed with PBS and changed into experimental medium containing a 1:1 mix of glutamine-free DMEM and glutamine-free Neurobasal medium including 10% dialyzed FBS, 2-mercaptoethanol, LIF, and 2 mM L-glutamine. The next day, cells were washed with PBS and changed into medium containing a 1:1 combination of glucose- and glutamine-free DMEM (Gibco) and glucose- and glutamine-free Neurobasal-A medium (ThermoFisher A24775-01) supplemented with ¹²C-glucose (Sigma) and ¹²C-glutamine (Gibco) or the ¹³C versions of each metabolite, [U-¹³C]glucose or [U-¹³C]glutamine (Cambridge Isotope Labs) to a final concentration of 20 mM (glucose) and 2 mM (glutamine). Enrichment of ¹³C was assessed by quantifying the abundance of the following ions: aKG, m/z 304-318; aspartate, m/z 334-346; citrate, m/z 465-482; fumarate, m/z 245-254; glutamate, m/z 363-377 and malate, m/z 335-347. Correction for natural isotope abundance was performed using IsoCor software (Millard et al., Bioinformatics 28, 1294-1296 (2012)).

### Immunofluorescence and Microscopy

For mouse ESCs, cells were seeded on 12-well MatTek glass-bottom dishes (P12G-1.0-10-F*) coated in laminin (Sigma, 10 μg/mL in PBS containing Ca²⁺ and Mg²⁺). Cells were fixed in 2% paraformaldehyde for 10 min and then permeabilized in 0.1% tween. Cells were washed with PBS and blocked for 1 h in 2.5% BSA in PBS. After blocking, cells were incubated overnight with primary antibodies diluted in blocking solution. The following antibodies were used: Oct3/4 (Santa Cruz Biotechnologies, sc-5279 at 1:100) and Nanog (eBioscience, 145761-80 at 1:125). The next day, cells were washed with PBS and incubated with secondary antibodies (AlexaFluor 488 or 594 or 647, Molecular Probes) diluted 1:500 in blocking solution for 1 h. For nuclear counterstaining, Hoechst 33342 (Molecular Probes, H3570 at 1 μg/mL) was added to the same secondary solution. After washing with PBS, cells were stored in the dark and imaged within 1 or 2 days. Cells were imaged using an AxioObserver.Z1 epifluorescence inverted microscope with a motorized stage. A CCD attached camera allowed digital image acquisition (Hammamatsu, Orca II). For multi-well and multidimensional microscopy, definite focus was used, and the microscope was programmed to image consecutive image fields (typically 60 per condition). These fields were stitched together using the built-in Axiovision function and exported as raw 16-bit TIFF files without further processing. Typically, at most 10,000 cells per well at 200× magnification were imaged.

Image analysis. Image analysis required three steps: cell detection, nuclear segmentation and fluorescence detection in a per cell basis. These steps were implemented on custom-made Matlab (MathWorks) routines. First, cells were detected by adapting a Matlab implementation of the IDL particle tracking code developed by David Grier, John Crocker, and Eric Weeks (http://physics.georgetown.edu/matlab/). This algorithm finds cells as peaks in a Fourier space rather than by thresholding. This approach is less susceptible to problems that typically arise when segmenting large images such as autofluorescent and bright speckles or day-to-day variability in imaging conditions. Cell detection allowed us to count, identify and get the spatial coordinates (centroid) for each cell. Second, nuclear segmentation was achieved by a combination of regular thresholding together with a watershed process based on the distance of cell centroids determined in the previous step. Obtained nuclear regions were then used as masks to quantify pixel intensities for all the fluorescent channels (that reported levels of different proteins) on a per cell basis. The present disclosure used cumulative values, which were then normalized to the Hoechst staining to correct for area, cell location along the Z-axis and DNA condensation differences. After image analysis, data was processed and plotted also with Matlab. Raw data, image analysis, and data processing routines are available upon request.

### Quantification of Gene Expression

RNA was isolated from six-well plates using Trizol (Invitrogen) according to manufacturer instructions. 200 ng RNA was used for cDNA synthesis using iScript (BioRad). Quantitative real-time PCR analysis was performed in technical triplicate using QuantStudio 7 Flex (Applied Biosystems) with Power SYBR Green (Life Technologies). All data were generated using cDNA from triplicate wells for each condition. Actin was used as an endogenous control for all experiments. The following primers were used:

Actin,
forward,
(SEQ ID NO. 1)
5′-GCTCTTTTCCAGCCTTCCTT-3′,
reverse,
(SEQ ID NO. 2)
5′-CTTCTGCATCCTGTCAGCAA-3′
Nanog,
forward,
(SEQ ID NO. 3)
5′-AAGATGCGGACTGTGTTCTC-3′,
reverse,
(SEQ ID NO. 4)
5′-CGCTTGCACTTCATCCTTTG-3′
Stat3,
forward,
(SEQ ID NO. 5)
5′-GTCCTTTTCCACCCAAGTGA-3′,
reverse,
(SEQ ID NO. 6)
5′-TATCTTGGCCCTTTGGAATG-3′
Tfcp2l1,
forward,
(SEQ ID NO. 7)
5′-GGGGACTACTCGGAGCATCT-3′,
reverse,
(SEQ ID NO. 8)
5′-TTCCGATCAGCTCCCTTG-3′;
Esrrb,
forward,
(SEQ ID NO. 9)
5′-AACAGCCCCTACCTGAACCT-3′,
reverse,
(SEQ ID NO. 10)
5′-TGCCAATTCACAGAGAGTGG-3′;
Klf4,
forward,
(SEQ ID NO. 11)
5′-CGGGAAGGGAGAAGACACT-3′,
reverse,
(SEQ ID NO. 12)
5′-GAGTTCCTCACGCCAACG-3′;
Zfp42,
forward,
(SEQ ID NO. 13)
5′-TCCATGGCATAGTTCCAACAG-3′,
reverse,
(SEQ ID NO. 14)
5′-TAACTGATTTTCTGCCGTATGC-3′.

### Western Blotting.

Protein lysates were extracted in 1× radioimmunoprecipitation assay buffer (Cell Signaling Technology), separated by SDS—polyacrylamide gel electrophoresis and transferred to nitrocellulose membranes (Bio-Rad). Membranes were blocked in 3% milk in Tris-buffered saline with 0.1% Tween20 (TBST) and incubated at 4° C. with primary antibodies overnight. After TBST washes the next day, membranes were incubated with horseradish peroxidase-conjugated secondary antibodies for 2 h, incubated with enhanced chemiluminescence (Thermo Fisher Scientific) and imaged with an SRX-101A X-ray Film Processor (Konica Minolta). The antibodies used (at 1:1,000 unless otherwise stated) were: Nanog (catalogue no. AF2729; R&D Systems); KLF4 (catalogue no. ab129473; Abcam), phospho-Stat3 (catalogue no. 9138; Cell Signaling Technology); Stat3 (124146, catalogue no. 9139; Cell Signaling Technology), and α-Tubulin (1:10,000, catalogue no. 79026; Sigma-Aldrich).

### Reprogramming of Mouse Embryonic Fibroblasts to Induced Pluripotent Stem Cells

Assessment of colony formation. Collagen-OKSM MEFs which contain an optimized reverse tetracycline-dependent transactivator (M2-rtTA) targeted to the constitutively active Rosa26 locus (https://www.jax.org/strain/006965) and a polycistronic cassette encoding Oct4, Klf4, Sox2, and c-Myc targeted to the Col1a1 locus under control of a tetracycline-dependent minimal promoter (tetOP) (Stadtfeld et al., Nat Methods 7, 53-55 (2010)) were plated at 50,000 cells per plate on gelatin-coated 6-well plates. 24 hours later, cells were washed with PBS and changed to S/L medium containing 1 μg/mL of doxycycline. Medium was replaced every 2 days. After 8 days of culture in S/L medium containing doxycycline, cells were washed with PBS and changed into experimental medium containing a 1:1 mix of glutamine-free DMEM and glutamine-free Neurobasal medium including 10% dialyzed FBS, 2-mercaptoethanol, LIF, and 2 mM L-glutamine without doxycycline. On day 10, all cells were washed with PBS and changed into experimental medium containing 2 mM L-glutamine (“Ctrl”), no glutamine (“Pulse”) or 2 mM L-glutamine plus 3 μM CHIR99021 (Stemgent) and 1 μM PD0325901 (Stemgent) (“2i” and “Pulse 2i”). 24 hours later, all cells were washed with PBS and returned to experimental medium containing 2 mM L-glutamine. “2i continuous” treated samples were supplemented with 2i from day 10 for the duration of the experiment. After 14 days, cells were stained for alkaline phosphatase expression and manually scored for colony formation in a blinded fashion as described above.

Assessment of Oct4-GFP expression. MEFs containing a GFP allele targeted to the endogenous Oct4/Pou5f1 locus (www.jax.org/strain/008214) (Lengner et al., Cell Stem Cell 1, 403-415 (2007)) were plated at 20,000 cells per well on gelatin-coated 6-well plates in DMEM medium containing 10% FBS. 24 hours later, cells were infected with lentivirus containing Oct4, Sox2, Klf4; and c-Myc under control of the tetracycline operator and a CMV promoter (a gift from Rudolf Jaenisch, Addgene plasmid #20321). 24 hours after infection, plates were washed with PBS and changed to standard S/L medium containing 1 μg/mL of doxycycline. Medium was replaced every 2 days. After 12 days of culture in S/L medium containing doxycycline, cells were washed with PBS and changed into experimental medium containing a 1:1 mix of glutamine-free DMEM and glutamine-free Neurobasal medium including 10% dialyzed FBS, 2-mercaptoethanol, LIF, and 2 mM L-glutamine without doxycycline. On day 14, all cells were washed with PBS and changed into experimental medium containing 2 mM L-glutamine (“Ctrl”), no glutamine (“Pulse”) or 2 mM L-glutamine plus 3 μM CHIR99021 (Stemgent) and 1 μM PD0325901 (Stemgent) (“2i”, “Pulse 2i”). 24 hours later, all cells were washed with PBS and returned to experimental medium containing 2 mM L-glutamine. “2i continuous” treated samples were supplemented with 2i from day 14 for the duration of the experiment. On day 20, cells were trypsinzed, resuspended in FACS buffer containing DAPI and assessed for GFP expression by flow cytometry as described above. Oct4-GFP positivity was defined by expression of GFP at most 10-fold higher than that of negative cells.

### Teratomas

ESCs were initially plated in standard serum/LIF medium as described earlier. The following day, cells were washed with PBS and then changed to experimental medium containing a 1:1 mix of glutamine-free DMEM and glutamine-free Neurobasal medium including 10% dialysed FBS, 2-mercaptoethanol and LIF, and containing (Ctrl) or lacking (Pulse—glutamine) l-glutamine as indicated; 24 h later, cells were washed with PBS and then returned to glutamine-replete medium (Recover). Twenty-four hours later, 1×106 cells per replicate were collected from each group and mixed 1:1 with medium plus Matrigel Basement Membrane Matrix (BD Biosciences) and injected into the flanks of recipient female SCID littermate mice aged 8-12 weeks (NOD scid gamma, stock no. JAX 005557; The Jackson Laboratory). All conditions produced tumours in 4-8 weeks. Mice were euthanized before tumour size exceeded 1.5 cm in diameter. Tumours were excised and fixed in 4% paraformaldehyde overnight at 4° C. Tumours were paraffin-embedded and sections were stained with haematoxylin and eosin according to standard procedures by HistoWiz. All experiments were performed in accordance with a protocol approved by the Memorial Sloan Kettering Institutional Animal Care and Use Committee.

### Statistical Analyses

GraphPad PRISM 7 software was used for statistical analyses except for IF data. Error bars, P values and statistical tests are reported in figure legends. Statistical analyses on images were performed using Matlab. The present disclosure set the threshold to define “Oct4-low” cells as one standard deviation below the mean values of the control population (typically S/L in the presence of glutamine).

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes

1. A method for selectively enriching pluripotent cells in a cell population comprising non-pluripotent cells and the pluripotent cells, wherein the method comprises culturing the cell population in a glutamine-deficient medium.

2. The method of claim 1, wherein the pluripotent cells are self-renewing pluripotent cells.

3. A method for selectively enriching fully reprogrammed pluripotent cells in a cell population comprising not fully reprogrammed cells and the fully reprogrammed pluripotent cells, wherein the method comprises culturing the cell population in a glutamine-deficient medium.

4. The method of claim 3, wherein the cell population are derived from somatic cells, wherein the somatic cells have been subject to reprogramming to induce acquired pluripotency.

5. The method claim 1, wherein the cell population is cultured in the glutamine-deficient medium transiently.

6. The method of claim 1, wherein the cell population is cultured in the glutamine-deficient medium for between about 4 hours and about 48 hours.

7. (canceled)

8. (canceled)

9. (canceled)

10. (canceled)

11. (canceled)

12. The method of claim 1, wherein the level of the pluripotent cells is increased between about 10% to about 500% as compared to the level of pluripotent cells in a cell population that has not been cultured in the glutamine-deficient medium.

13. (canceled)

14. The method of claim 1, wherein the pluripotent cells has an elevated cellular αKG/succinate ratio as compared to the non-pluripotent cells and/or a high level of Nanog, Oct4, Sox2, Esrrb, Zfp42, Klf4, Tfcp2l1, Stat3, or combinations thereof as compared to the non-pluripotent cells.

15. (canceled)

16. (canceled)

17. A plurality of pluripotent cells, wherein the pluripotent cells are selectively enriched in a cell population comprising non-pluripotent cells and the pluripotent cells, after culturing the cell population in a glutamine-deficient medium.

18. The pluripotent cells of claim 17, wherein the pluripotent cells are self-renewing pluripotent cells.

19. (canceled)

20. The pluripotent cells of claim 17, wherein the cell population is cultured in the glutamine-deficient medium for between about 4 hours and about 48 hours.

21. (canceled)

22. (canceled)

23. (canceled)

24. (canceled)

25. (canceled)

26. The pluripotent cells of claim 17, wherein the level of the pluripotent cells in the cell population is increased between about 10% to about 500% as compared to the level of pluripotent cells in a cell population that has not been cultured in the glutamine-deficient medium.

27. (canceled)

28. The pluripotent cells of claim 17, wherein the pluripotent cells has an elevated cellular αKG/succinate ratio and/or a high level of Nanog, Oct4, Sox2, Esrrb, Zfp42, Klf4, Tfcp2l1, Stat3, or combinations thereof as compared to the non-pluripotent cells as compared to the non-pluripotent cells.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A composition comprising the pluripotent cells of claim 17.

43. (canceled)

44. A kit for selectively enriching pluripotent cells, comprising: a glutamine-deficient medium, and a cell population comprising non-pluripotent cells and the pluripotent cells.

45. The kit of claim 44, wherein the pluripotent cells are self-renewing pluripotent cells.

46. (canceled)

47. (canceled)

48. (canceled)

49. (canceled)

50. (canceled)

51. (canceled)

52. (canceled)

53. (canceled)

54. (canceled)

55. (canceled)

56. (canceled)


SEQUENCE LISTING (14 sequences)

SEQ ID NO: 1 - DNA, 20 bp, Artificial Sequence
  Synthetic polynucleotide
gctcttttccagccttcctt

SEQ ID NO: 2 - DNA, 20 bp, Artificial Sequence
  Synthetic polynucleotide
cttctgcatcctgtcagcaa

SEQ ID NO: 3 - DNA, 20 bp, Artificial Sequence
  Synthetic polynucleotide
aagatgcggactgtgttctc

SEQ ID NO: 4 - DNA, 20 bp, Artificial Sequence
  Synthetic polynucleotide
cgcttgcacttcatcctttg

SEQ ID NO: 5 - DNA, 20 bp, Artificial Sequence
  Synthetic polynucleotide
gtccttttccacccaagtga

SEQ ID NO: 6 - DNA, 20 bp, Artificial Sequence
  Synthetic polynucleotide
tatcttggccctttggaatg

SEQ ID NO: 7 - DNA, 20 bp, Artificial Sequence
  Synthetic polynucleotide
ggggactactcggagcatct

SEQ ID NO: 8 - DNA, 18 bp, Artificial Sequence
  Synthetic polynucleotide
ttccgatcagctcccttg

SEQ ID NO: 9 - DNA, 20 bp, Artificial Sequence
  Synthetic polynucleotide
aacagcccctacctgaacct

SEQ ID NO: 10 - DNA, 20 bp, Artificial Sequence
  Synthetic polynucleotide
tgccaattcacagagagtgg

SEQ ID NO: 11 - DNA, 19 bp, Artificial Sequence
  Synthetic polynucleotide
cgggaagggagaagacact

SEQ ID NO: 12 - DNA, 18 bp, Artificial Sequence
  Synthetic polynucleotide
gagttcctcacgccaacg

SEQ ID NO: 13 - DNA, 21 bp, Artificial Sequence
  Synthetic polynucleotide
tccatggcatagttccaacag

SEQ ID NO: 14 - DNA, 22 bp, Artificial Sequence
  Synthetic polynucleotide
taactgattttctgccgtatgc